(19) United States
(12) Patent Application Publication
(10) Pub. No.: US 2014/0024638 A1
(43) Pub. Date: Jan. 23, 2014

(54) TREATMENT OF COGNITIVE DYSFUNCTION IN SCHIZOPHRENIA

(71) Applicants: Merouane Bencherif, Winston-Salem, NC (US); Geoffrey C. Dunbar, Winston-Salem, NC (US); David A. Hosford, Durham, NC (US); Gregory J. Gatto, Winston-Salem, NC (US); Terry Hauser, Winston-Salem, NC (US); Kristen G. Jordan, Clemmons, NC (US); Anthony Carl Segreti, Cary, NC (US)

(72) Inventors: Merouane Bencherif, Winston-Salem, NC (US); Geoffrey C. Dunbar, Winston-Salem, NC (US); David A. Hosford, Durham, NC (US); Gregory J. Gatto, Winston-Salem, NC (US); Terry Hauser, Winston-Salem, NC (US); Kristen G. Jordan, Clemmons, NC (US); Anthony Carl Segreti, Cary, NC (US)

(73) Assignee: TARGACEPT, INC., Winston-Slaem, NC (US)

(21) Appl. No.: 13/980,091

(22) PCT Filed: Jan. 17, 2012
(86) PCT No.: PCT/US12/21472

§ 371 Date: Oct. 7, 2013

Related U.S. Application Data
(60) Provisional application No. 61/467,278, filed on Mar. 24, 2011.
(60) Provisional application No. 61/438,872, filed on Feb. 2, 2011.
(60) Provisional application No. 61/433,795, filed on Jan. 18, 2011.

(51) Int. Cl. A61K 31/444; A61K 45/06

(52) U.S. Cl. CPC A61K 31/444; A61K 45/06
USPC 514/211.13; 514/305; 514/225.8; 514/252.13; 514/226.2; 514/253.07; 514/254.04; 514/220

(57) ABSTRACT

The present invention relates to (2S,3R)—N-(2-((3-pyridinyl)methyl)-1-azabicyclo[2.2.2]oct-3-yl)benzofuran-2-carboxamide or a pharmaceutically acceptable salt thereof, pharmaceutical compositions, and methods for treating schizophrenia.

### FIELD OF THE INVENTION

The present invention relates to methods and uses for (2S,3R)—N-(2-((3-pyridinyl)methyl)-1-azabicyclo[2.2.2]oct-3-yl)benzofuran-2-carboxamide or a pharmaceutically acceptable salt thereof.

### BACKGROUND

The compound (2S,3R)—N-(2-((3-pyridinyl)methyl)-1-azabicyclo[2.2.2]oct-3-yl)benzofuran-2-carboxamide, is described, including methods of synthesis, in U.S. Pat. No. 7,981,906 (Application Publication No. US 2009/0048290 A1), and is part of a genus described in U.S. Pat. No. 6,953,855, both of which are fully incorporated herein by reference. The compound may be referred to as TC-5619.

Schizophrenia is a chronic, severe, and disabling form of psychosis. In addition to symptoms such as delusions, hallucinations, the inability to disregard familiar stimuli (sometimes referred to as sensory gating), disorganized speech, grossly disorganized or catatonic behavior and prolonged loss of emotion, feeling, volition or drive, schizophrenia is often marked by impairment in cognitive functions, such as executive function, attention, vigilance, memory and reasoning. These cognitive impairments play a primary role in the inability of schizophrenic patients to function normally. It has been estimated that up to 75% of persons with schizophrenia are cognitively impaired (derived based on a reported prevalence of schizophrenia of 4.6 million in the world's seven major pharmaceutical markets in 2009 (Patient Base, a database of epidemiology available through Decision Resources, Inc., May 2010), and an estimate of 75% of schizophrenics that have cognitive dysfunction (O'Carroll, R., Cognitive impairment in schizophrenia. Advances in Psychiatric Treatment, 2000)). There is currently no drug approved in the United States or Europe specifically for cognitive dysfunction in schizophrenia.

As will be provided in more detail herein, one measure of clinical efficacy for cognitive dysfunction in schizophrenia includes the Schizophrenia Cognitive Test Battery. Reference is made to Collie A, Maruff P, Snyder P J. (2006) Does atypical antipsychotic medication improve executive function in schizophrenia? Int J Neuropsychopharmacol. 9, 629-630; author reply 631-632; Falleti M G, Maruff P, Collie A, Darby D G. (2006). Practice effects associated with the repeated assessment of cognitive function using the CogState battery at 10-minute, one week and one month test-retest intervals. J Clin Exp Neuropsychol. 28, 1095-1112; Snyder P J, Piskulic D, Olver J, Norman T, Maruff P. (2006). Spatial working memory and problem solving in schizophrenia: The effect of symptom stabilization with atypical antipsychotic medication. Psychiatry Research. In press; Snyder P J, O'Sullivan R, Jackson C, Olver J, Norman T, Piskulic D, Collie A, Maruff P. (2006). Stability of cognitive performance in chronic schizophrenia over brief and immediate re-test intervals: Implications for studies of treatment efficacy. Human Psychopharmacology. In press; and (http://www.cogstate.com/go/clifficaltrials/our-test-batteries/schizophrenia-battery; CogState, New Haven, Conn.).

Schizophrenia can be divided into three major phases: the prodromal state, an active phase, and a residual phase. These phases tend to occur in sequence and appear in cycles throughout the course of the illness.

During the prodromal state it is not uncommon for a number of non-specific symptoms to be present in the weeks or months preceding the first onset of typical symptoms of schizophrenia, particularly in young people. These symptoms include:
A general loss of interest;
Avoidance of social interactions;
Avoidance of work or study (e.g. dropping out of school, college or university);
Being irritable and oversensitive;
Odd beliefs (e.g. superstitiousness); and
Odd behavior (e.g. talking to self in public).

These changes will often be incapacitating for the individual and distressing for the family. Friends or relatives may describe the individual as “no longer the same person”. The length of the prodromal phase is extremely variable and prognosis is less favorable when the prodromal phase has had a lengthy course. When symptoms develop gradually, people may begin to lose interest in their usual pursuits and to withdraw from friends and family members. They may become easily confused, have trouble concentrating, and feel listless and apathetic, preferring to spend most of their days alone. They may also become intensely preoccupied with religion or philosophy. Family and friends may be upset with this behavior, believing the person is lazy rather than ill. Occasionally, these symptoms reach a plateau and do not develop further but, in most cases, an active phase of the illness follows. The prodromal period can last weeks or months. Although the symptoms described above are typical of the prodromal phase of schizophrenia, they may also be due to other causes.

During the active phase of the illness, psychotic symptoms such as delusions, odd behavior and hallucinations are prominent and are often accompanied by strong affect such as distress, anxiety, depression, and fear. If untreated, the active phase may resolve spontaneously or may continue indefinitely. With appropriate treatment (primarily medication) the active phase is usually able to be brought under control. It is during the active phase that most individuals present for treatment, whether it is their first presentation or an exacerbation of their symptoms. During schizophrenia's active phase, people may experience delusions, hallucinations, marked distortions in thinking and disturbances in behavior and feelings. This phase most often appears after a prodromal period. On occasion, these symptoms can appear suddenly.

The active phase of the illness is usually followed by a residual phase. The residual phase is similar to the prodromal phase although during the residual phase blunted affect and impairment in role functioning are more common. While psychotic symptoms may persist into the residual phase, the psychotic symptoms are less likely to be accompanied by such strong affect as experienced during the active phase. There is great variation in the severity of the residual phase from one person to the next. Some individuals will function extremely well while others may be considerably more impaired. After an active phase, people may be listless, have trouble concentrating and be withdrawn. The symptoms in this phase are similar to those outlined under the prodromal phase. If there have been no symptoms before the first episode, few or no symptoms may be experienced afterward. During a lifetime; people with schizophrenia may become actively ill once or twice, or have many more episodes. Unfortunately, residual symptoms may increase, while ability to function normally may decrease, after each active phase. It is therefore important to try to avoid relapses by following the prescribed treatment. Currently it is difficult to predict at the onset how fully a person will recover.

Reference is made to Marder et al., Methodological Issues in Negative Symptom Trials, Schizophrenia Bulletin, 2011; and Laughren and Levin, Food and Drug Administration Commentary of Methodological Issues in Negative Symptom Trials, Schizophrenia Bulletin, 2011.

The most common course of the disorder generally involves numerous active phases of illness with residual phases of impairment between episodes. The extent of residual impairment often increases between episodes during the initial years of the disorder although may possibly become less severe during the later phases of the illness.

### SUMMARY OF THE INVENTION

In one aspect, the invention relates to the treatment of cognitive dysfunction in schizophrenia by administering (2S,3R)—N-(2-((3-pyridinyl)methyl)-1-azabicyclo[2.2.2]oct-3-yl)benzofuran-2-carboxamide or a pharmaceutically acceptable salt thereof to a patient in need thereof. In an embodiment, the invention relates to treatment of cognitive dysfunction in schizophrenia by improving executive function. In another embodiment, the invention relates to treatment of cognitive dysfunction in schizophrenia by improving memory. In another embodiment, the invention relates to treatment of cognitive dysfunction in schizophrenia by improving attention.

In another aspect, the invention relates to treatment of negative symptoms of schizophrenia by administering (2S,3R)—N-(2-((3-pyridinyl)methyl)-1-azabicyclo[2.2.2]oct-3-yl)benzofuran-2-carboxamide or a pharmaceutically acceptable salt thereof to a patient in need thereof.

In another aspect, the invention relates to the treatment of residual phase schizophrenia.

Various terms used herein, not otherwise defined, may be defined with reference to the Protocol.

### BRIEF DESCRIPTION OF THE INVENTION

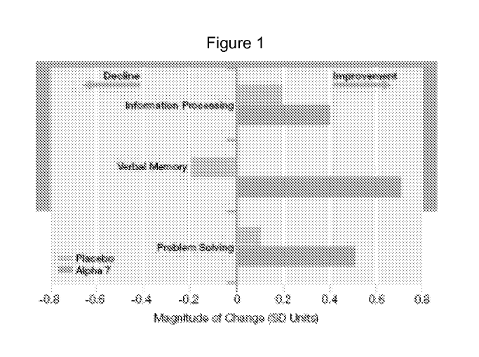

FIG. 1 is a graphical depiction that summarizes the statistically significant results from a placebo controlled parallel group study comparing the effect of a novel alpha 7 nicotine agonist on the Simple Reaction Time (information processing), International Shopping List (verbal learning) and Groton Maze Learning Task (problem solving) tasks in patients with chronic schizophrenia (n=30 per group) who were stable on their antipsychotic medication at randomization. Improvement in performance of greater than 0.4 standard deviation units was found for each measure.

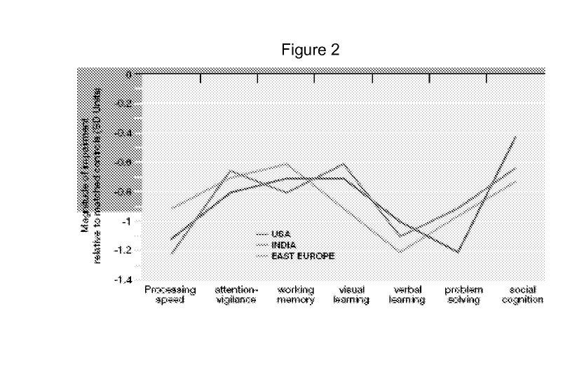

FIG. 2 illustrates sensitivity of the primary outcome measures from the CogState schizophrenia battery to cognitive impairment in patients with chronic schizophrenia who were receiving antipsychotic medication in three different geographical areas. The nature and magnitude of impairment in the different cognitive domains was consistent across the three cultural groups.

### DETAILED DESCRIPTION OF THE INVENTION

(2S,3R)—N-(2-((3-pyridinyl)methyl)-1-azabicyclo[2.2.2]oct-3-yl)benzofuran-2-carboxamide or a pharmaceutically acceptable salt thereof, (2S,3R)—N-(2-((3-pyridinyl)methyl)-1-azabicyclo[2.2.2]oct-3-yl)benzofuran-2-carboxamide, is described, including methods of synthesis, in U.S. Pat. No. 7,981,906 (previously published as Publication No. US 2009/0048290 A1), and is part of a genus described in U.S. Pat. No. 6,953,855, both of which are fully incorporated herein by reference.

(2S,3R)—N-(2-((3-pyridinyl)methyl)-1-azabicyclo[2.2.2]oct-3-yl)benzofuran-2-carboxamide or a pharmaceutically acceptable salt thereof is a selective alpha7 NNR agonist. (2S,3R)—N-(2-((3-pyridinyl)methyl)-1-azabicyclo[2.2.2]oct-3-yl)benzofuran-2-carboxamide or a pharmaceutically acceptable salt thereof had efficacy in preclinical models of memory and was generally well tolerated in phase 1 trials in healthy volunteers, who demonstrated a robust improvement in attention when 6.7 mg (2S,3R)—N-(2-((3-pyridinyl)methyl)-1-azabicyclo[2.2.2]oct-3-yl)benzofuran-2-carboxamide or a pharmaceutically acceptable salt thereof was administered. See, Hauser T A, Kucinski A, Jordan K G, Gatto G J, Lippiello P M, Bencherif M: (2S,3R)—N-(2-((3-pyridinyl)methyl)-1-azabicyclo[2.2.2]oct-3-yl)benzofuran-2-carboxamide or a pharmaceutically acceptable salt thereof: An α7 NNR selective agonist that demonstrates efficacy in animal models of schizophrenia, Biochem. Pharmacol. 1009; 78: 803-812, herein incorporated by reference.

A Phase 2 clinical proof of concept trial was conducted to evaluate (2S,3R)—N-(2-((3-pyridinyl)methyl)-1-azabicyclo[2.2.2]oct-3-yl)benzofuran-2-carboxamide or a pharmaceutically acceptable salt thereof as an augmentation therapy to improve cognition in patients with schizophrenia. In the trial, (2S,3R)—N-(2-((3-pyridinyl)methyl)-1-azabicyclo[2.2.2]oct-3-yl)benzofuran-2-carboxamide or a pharmaceutically acceptable salt thereof met the protocol criteria for a positive result on the primary efficacy outcome measure, the Groton Maze Learning Task (GMLT) of the CogState Schizophrenia Battery (CogState, New Haven, Conn.), and was well tolerated. The trial is Study Number PRO-05619-CRD-001, incorporated herein below.

The statistically significant and qualitatively similar effects favoring (2S,3R)—N-(2-((3-pyridinyl)methyl)-1-azabicyclo[2.2.2]oct-3-yl)benzofuran-2-carboxamide or a pharmaceutically acceptable salt thereof in the primary objective endpoint (GMLT), in a number of secondary clinician and patient-rated endpoints (SANS, CGI-Global, and SGI-Cog), and in CogState objective cognitive endpoints underscore the positive efficacy of (2S,3R)—N-(2-((3-pyridinyl)methyl)-1-azabicyclo[2.2.2]oct-3-yl)benzofuran-2-carboxamide or a pharmaceutically acceptable salt thereof in Cognitive Deficits in Schizophrenia.

(2S,3R)—N-(2-((3-pyridinyl)methyl)-1-azabicyclo[2.2.2]oct-3-yl)benzofuran-2-carboxamide or a pharmaceutically acceptable salt thereof exhibited a favorable tolerability profile in the trial, and there was no clinically significant difference between the (2S,3R)—N-(2-((3-pyridinyl)methyl)-1-azabicyclo[2.2.2]oct-3-yl)benzofuran-2-carboxamide or a pharmaceutically acceptable salt thereof and placebo dose groups in discontinuations due to adverse events. The most frequent adverse event that was more common in the (2S,3R)—N-(2-((3-pyridinyl)methyl)-1-azabicyclo[2.2.2]oct-3-yl)benzofuran-2-carboxamide or a pharmaceutically acceptable salt thereof cohort than in the placebo cohort was nausea (0% placebo vs. 5% (2S,3R)—N-(2-((3-pyridinyl)methyl)-1-azabicyclo[2.2.2]oct-3-yl)benzofuran-2-carboxamide or a pharmaceutically acceptable salt thereof), which was mild to moderate in severity and never led to patient dropout. There were two serious adverse events in the trial, one in the placebo dose group and one in the (2S,3R)—N-(2-((3-pyridinyl)methyl)-1-azabicyclo[2.2.2]oct-3-yl)benzofuran-2-carboxamide or a pharmaceutically acceptable salt thereof dose group. Both were considered by the applicable investigator as not drug related.

The efficacy of (2S,3R)—N-(2-((3-pyridinyl)methyl)-1-azabicyclo[2.2.2]oct-3-yl)benzofuran-2-carboxamide or a pharmaceutically acceptable salt thereof against negative symptoms and cognitive symptoms is a remarkable finding due to the relative lack of effect of atypical antipsychotics upon these residual symptoms of schizophrenia. Because these residual symptoms are the primary reason that people with schizophrenia do not regain their full pre-morbid level of function, a new treatment for these symptoms fills a major unmet need. This need has been recognized by the NIMH through their MATRICS initiative (Neuchterlein et al., 2004; Gold, 2004), other initiatives with broad academic and regulatory support (Blanchard et al., 2010; Marder et al., 2011), and endorsed by the FDA (Laughren and Levin, 2011).

The MATRICS initiative has highlighted the potential for small molecules that target the alpha7 NNR receptor in the treatment for cognitive dysfunction in schizophrenia. That potential was supported by preclinical models of schizophrenia in which the alpha 7 NNR agonist, (2S,3R)—N-(2-((3-pyridinyl)methyl)-1-azabicyclo[2.2.2]oct-3-yl)benzofuran-2-carboxamide or a pharmaceutically acceptable salt thereof, was effective; and by early clinical studies in which a variety of other alpha7 NNR agonists were effective against surrogate markers (Olincy et al., 2006; EnVivo Pharmaceuticals, 2009) and measured features of schizophrenia (Freedman et al., 2008).

### I. Compound

The compound of the present invention is (2S,3R)—N(2-((3-pyridinyl)methyl)-1-azabicyclo[2.2.2]oct-3-yl)benzofuran-2-carboxamide, represented as Compound A below, or a pharmaceutically acceptable salt forms of Compound A.

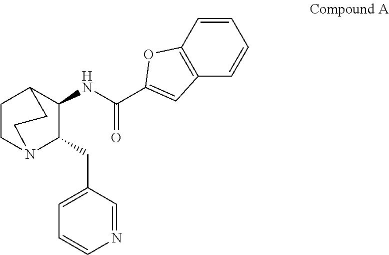 [SMILES: O=C(N[C@@H]1C2CCN(CC2)[C@H]1Cc1cccnc1)c1cc2ccccc2o1]

(2S,3R)—N(2-((3-Pyridinyl)methyl)-1-azabicyclo[2.2.2]oct-3-yl)benzofuran-2-carboxamide (Compound A) is a highly selective, full agonist at the α7 NNR receptor with a remarkably low EC₅₀ (for activation) value and a good separation between EC₅₀ and the IC₅₀ (for residual inhibition), providing functional agonism over a broad range of therapeutically useful concentrations.

### II. Scalable Synthesis of (2S,3R)—N-(2-((3-pyridinyl)methyl)-1-azabicyclo[2.2.2]oct-3-yl)benzofuran-2-carboxamide

Particular synthetic steps vary in their amenability to scale-up. Reactions are found lacking in their ability to be scaled-up for a variety of reasons, including safety concerns, reagent expense, difficult work-up or purification, reaction energetics (thermodynamics or kinetics), and reaction yield.

The synthesis of (2S,3R)—N-(2-((3-pyridinyl)methyl-1-azabicyclo[2.2.2]oct-3-yl)benzofuran-2-carboxamide described herein has been used to produce kilogram quantities of material, and the component reactions have been carried out on multi-kilogram scale in high yield.

The scalable synthesis utilizes both the dynamic resolution of a racemizable ketone (2-((3-pyridinyl)methyl)-1-azabicyclo[2.2.2]octan-3-one) and the stereoselective reduction of the (R)-α-methylbenzylamine imine derivative (reductive amination) of the resolved ketone. The synthetic sequences reported herein are readily scalable and avoid chromatographic purifications.

### III. Preparation of Salt Forms of (2S,3R)—N-(2-((3-pyridinyl)methyl-1-azabicyclo[2.2.2]oct-3-yl)benzofuran-2-carboxamide

(2S,3R)—N-(2-((3-Pyridinyl)methyl-1-azabicyclo[2.2.2]oct-3-yl)benzofuran-2-carboxamide as a free base is an amorphous powder with very limited water solubility. The free base will react with both inorganic and organic acids to make certain acid addition salts that have physical and chemical properties that are advantageous for the preparation of pharmaceutical compositions, including but not limited to crystallinity, water solubility, and stability. The stoichiometry of the salts of the present invention can vary.

Depending upon the manner by which the salts described herein are formed, the salts can have crystal structures that occlude solvents that are present during salt formation. Thus, the salts can occur as hydrates and other solvates of varying stoichiometry of solvent relative to the (2S,3R)—N-(2-((3-pyridinyl)methyl-1-azabicyclo[2.2.2]oct-3-yl)benzofuran-2-carboxamide.

The method for preparing the salt forms can vary. The preparation of (2S,3R)—N-(2-((3-pyridinyl)methyl-1-azabicyclo[2.2.2]oct-3-yl)benzofuran-2-carboxamide salt forms generally involves: (i) mixing the free base or a solution of the free base, namely (2S,3R)—N-(2-((3-pyridinyl)methyl-1-azabicyclo[2.2.2]oct-3-yl)benzofuran-2-carboxamide in a suitable solvent with an acid neat, or as a solution of an acids in a suitable solvent; (iia) cooling the resulting salt solution, if necessary to cause precipitation; or (iib) adding a suitable anti-solvent to cause precipitation; or (iic) evaporating the first solvent and adding a new solvent and repeating either steps (iia) or step (iib); and (iii) filtering and collecting the resulting salt. The stoichiometry, solvent mix, solute concentration, and temperature employed can vary. Representative solvents that can be used to prepare or recrystallize the salt forms include, without limitation, ethanol, methanol, isopropyl alcohol, acetone, ethyl acetate, and acetonitrile.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; and salts with amino acids such as aspartate and glutamate. The salts may be in some cases hydrates or ethanol solvates. Representative salts are provided as described in U.S. Pat. Nos. 5,597,919 to Dull et al., 5,616,716 to Dull et al. and 5,663,356 to Ruecroft et al, each of which is incorporated by reference. Salt screening for the free base (2S,3R)—N-(2-((3-pyridinyl)methyl-1-azabicyclo[2.2.2]oct-3-yl)benzofuran-2-carboxamide revealed that, while many salts of pharmaceutically acceptable acids could be formed, only a few of these salts had acceptable properties for commercial manufacture. The ability to predict the characteristics exemplified by a commercially viable salt, therefore, does not exist. Acids that provided salts that were crystalline, namely salts that demonstrate some degree of crystallinity, dependent upon the method by which they are prepared, include hydrochloric acid, sulfuric acid, phosphoric acid, p-toluenesulfonic acid, galactaric (mucic) acid, D-mandelic acid, D-tartaric acid, methanesulfonic acid, R- and S-10-camphorsulfonic acids, maleic acid, ketoglutaric acid and hippuric acid. Of these salts, the hydrochloric acid, phosphoric acid, maleic acid and p-toluenesulfonic acid salts each exhibited additional desirable properties, including high melting points, good water solubility, and low hygroscopicity.

### IV. Pharmaceutical Compositions

The pharmaceutical compositions of the present invention include the salts described herein, in the pure state or in the form of a composition in which the compounds are combined with any other pharmaceutically compatible product, which can be inert or physiologically active. The resulting pharmaceutical compositions can be used to prevent a condition or disorder in a subject susceptible to such a condition or disorder, and/or to treat a subject suffering from the condition or disorder. The pharmaceutical compositions described herein include the compound of the present invention and/or pharmaceutically acceptable salts thereof.

The manner in which the compounds are administered can vary. The compositions are preferably administered orally (e.g., in liquid form within a solvent such as an aqueous or non-aqueous liquid, or within a solid carrier). Preferred compositions for oral administration include pills, tablets, capsules, caplets, syrups, and solutions, including hard gelatin capsules and time-release capsules. Standard excipients include binders, fillers, colorants, solubilizers, and the like. Compositions can be formulated in unit dose form, or in multiple or subunit doses. Preferred compositions are in liquid or semisolid form. Compositions including a liquid pharmaceutically inert carrier such as water or other pharmaceutically compatible liquids or semisolids can be used. The use of such liquids and semisolids is well known to those of skill in the art.

The compositions can also be administered via injection, i.e., intravenously, intramuscularly, subcutaneously, intraperitoneally, intraarterially, intrathecally; and intracerebroventricularly. Intravenous administration is the preferred method of injection. Suitable carriers for injection are well known to those of skill in the art and include 5% dextrose solutions, saline, and phosphate-buffered saline. The drug product can also be administered as an infusion or injection (e.g., as a suspension or as an emulsion in a pharmaceutically acceptable liquid or mixture of liquids).

The formulations can also be administered using other means, for example, rectal administration. Formulations useful for rectal administration, such as suppositories, are well known to those of skill in the art. The drug product can also be administered by inhalation (e.g., in the form of an aerosol either nasally or using delivery articles of the type set forth in U.S. Pat. No. 4,922,901 to Brooks et al., the disclosure of which is incorporated herein in its entirety); topically (e.g., in lotion form); transdermally (e.g., using a transdermal patch) or iontophoretically; or by sublingual or buccal administration. Although it is possible to administer a compound in the form of a bulk active chemical, it is preferred to present a drug product in the form of a pharmaceutical composition or formulation for efficient and effective administration.

Exemplary methods for administering compounds will be apparent to the skilled artisan. The usefulness of these formulations can depend on the particular composition used and the particular subject receiving the treatment. These formulations can contain a liquid carrier that can be oily, aqueous, emulsified or contain certain solvents suitable to the mode of administration.

The compositions can be administered intermittently or at a gradual, continuous, constant or controlled rate to a warm-blooded animal (e.g., a mammal such as a mouse, rat, cat, rabbit, dog, pig, cow, or monkey), but advantageously are administered to a human being. In addition, the time of day and the number of times per day that the pharmaceutical formulation is administered can vary.

Other suitable methods for administering the compounds of the present invention are described in U.S. Pat. No. 5,604,231 to Smith et al., the contents of which are hereby incorporated by reference.

In an embodiment of the present invention and as will be appreciated by those skilled in the art, the compound of the present invention may be administered in combination with other therapeutic compounds or alternative, supplemental therapies.

The American Psychiatric Association's Guideline For The Treatment Of Patients With Schizophrenia states: “antipsychotic medications are indicated for nearly all acute psychotic episodes in patients with schizophrenia.” In addition to antipsychotic medications, some patients also take anti-depressants or mood-stabilizers to help control related symptoms. One aspect of the present invention includes a combination of (2S,3R)—N-(2-((3-pyridinyl)methyl)-1-azabicyclo[2.2.2]oct-3-yl)benzofuran-2-carboxamide or a pharmaceutically acceptable salt thereof, with one or more antipsychotic medications, anti-depressants, or mood stabilizers.

One aspect of the present invention includes a combination of (2S,3R)—N-(2-((3-pyridinyl)methyl)-1-azabicyclo[2.2.2]oct-3-yl)benzofuran-2-carboxamide or a pharmaceutically acceptable salt thereof, with one or more of: Stelazine (Trifluoperazine), Flupenthixol (Fluanxol), Loxapine (Loxapac, Loxitane), Perphenazine (Etrafon, Trilafon), Chlorpromazine (Thorazine), Haldol (Haloperidol), Prolixin (Fluphenazine Decanoate, Modecate, Permitil), Atypical Medications for Schizophrenia including but not limited to: Aripiprazole (Abilify) Clozaril (clozapine), Geodon (ziprasidone), Risperdal (resperidone), Seroquel (Quetiapine), or Zyprexa (olanzapine), or one or more new agents including but not limited to D2/5-HT2 antagonists such as iloperidone, DU 127090, or ORG 5222, D3 antagonists such as DTA 201A, neurokinin-3 antagonists such as osanetant, or another agonist of the nicotinic α7 receptor such as MEM3454.

Although an important element, medication is not the only treatment used for schizophrenia patients. Many patients and their families choose supplemental therapies (these can include psychosocial or cognitive therapy, rehabilitation day programs, peer support groups, nutritional supplements, etc) to use in conjunction with their medications. In certain severe cases, some patients also respond to electroconvulsive therapy (which has been shown to be safe and effective) or transcranial magnetic stimulation (TMS). These additional may help a person manage depression, social interactions, school, work, and the components for a full life.

In the case of therapy, some research has shown that psychotherapy and medication can be more effective than medication alone (however, the same study noted that psychotherapy alone was not a substitute for medication). The three main types of psychosocial therapy are: behavioral therapy (focuses on current behaviors), cognitive therapy (focuses on thoughts and thinking patterns), and interpersonal therapy (focuses on current relationships). For schizophrenia, cognitive-behavioral therapy has shown the most promise in conjunction with medication. Another aspect of the present invention includes a combination of (2S,3R)—N-(2-((3-pyridinyl)methyl)-1-azabicyclo[2.2.2]oct-3-yl)benzofuran-2-carboxamide or a pharmaceutically acceptable salt thereof with one or more supplemental therapy.

The compounds of the present invention may be employed alone or in combination with other therapeutic agents. Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination may be by administration concomitantly in: (1) a unitary pharmaceutical composition including multiple compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The compounds of the present invention may be used in the treatment of a variety of disorders and conditions and, as such, the compounds of the present invention may be used in combination with a variety of other suitable therapeutic agents useful in the treatment or prophylaxis of those disorders or conditions.

The appropriate dose of the compound is that amount effective to prevent occurrence of the symptoms of the disorder or to treat some symptoms of the disorder from which the patient suffers. As noted, by “effective amount”, “therapeutic amount” or “effective dose” is meant that amount sufficient to elicit the desired pharmacological or therapeutic effects, thus resulting in effective prevention or treatment of the disorder.

When treating a CNS disorder such as schizophrenia, an effective amount of compound is an amount sufficient to pass across the blood-brain barrier of the subject, to bind to relevant receptor sites in the brain of the subject and to modulate the activity of relevant NNR subtypes (e.g., provide neurotransmitter secretion, thus resulting in effective prevention or treatment of the disorder). An example of prevention of a disorder is manifested by delaying the onset of the symptoms of the disorder. An example of treatment of a disorder is manifested by a decrease in the symptoms associated with the disorder or an amelioration of the recurrence of the symptoms of the disorder. Preferably, the effective amount is sufficient to obtain the desired result, but insufficient to cause appreciable side effects.

The effective dose can vary, depending upon factors such as the condition of the patient, the severity of the symptoms of the disorder, and the manner in which the pharmaceutical composition is administered. For human patients, the effective dose of typical compounds generally requires administering the compound in an amount sufficient to modulate the activity of relevant NNRs, but the amount should be insufficient to induce effects on skeletal muscles and ganglia to any significant degree. The effective dose of compounds will of course differ from patient to patient, but in general includes amounts starting where CNS effects or other desired therapeutic effects occur but below the amount where muscular effects are observed.

The compounds described herein, when employed in effective amounts in accordance with the methods described herein, can provide some degree of prevention of the progression of, ameliorate symptoms of, or ameliorate, to some degree, the recurrence of CNS or other disorders. The effective amounts of those compounds are typically below the threshold concentration required to elicit any appreciable side effects, for example those effects relating to skeletal muscle or ganglia. The compounds can be administered in a therapeutic window in which certain CNS and other disorders are treated and certain side effects are avoided. Ideally, the effective dose of the compounds described herein is sufficient to provide the desired effects upon the disorder but is insufficient (i.e., is not at a high enough level) to provide undesirable side effects. Preferably, the compounds are administered at a dosage effective for treating the CNS or other disorders but less than, often less than ⅕, and often less than 1/10, the amount required to elicit certain side effects to any significant degree.

Most preferably, effective doses are at very low concentrations, where maximal effects are observed to occur, with a minimum of side effects. Typically, the effective dose of such compounds generally requires administering the compound in an amount of less than 5 mg/kg of patient weight. Often, the compounds of the present invention are administered in an amount from less than about 1 mg/kg patent weight and usually less than about 100 μg/kg of patient weight, but frequently between about 10 μg to less than 100 μg/kg of patient weight. The foregoing effective doses typically represent that amount administered as a single dose, or as one or more doses administered over a 24-hour period. For human patients, the effective dose of typical compounds generally requires administering the compound in an amount of at least about 1, at least about 10, and at least about 100 mg/24 hr/patient. For human patients, the effective dose of typical compounds requires administering the compound which generally does not exceed about 500, often does not exceed about 400, and frequently does not exceed about 300 mg/24 hr/patient. In addition, the compositions are advantageously administered at an effective dose such that the concentration of the compound within the plasma of the patient normally does not exceed 150 ng/mL, often does not exceed 50 ng/mL, and frequently does not exceed 20 ng/mL. In one embodiment of the present invention, an effective dose is between about 1 mg and 50 mg in a 24-hour period.

Method of Using Pharmaceutical Compositions

As used herein, “intrinsic activity” or “efficacy” relates to some measure of biological effectiveness of the binding partner complex. With regard to receptor pharmacology, the context in which intrinsic activity or efficacy should be defined will depend on the context of the binding partner (e.g., receptor/ligand) complex and the consideration of an activity relevant to a particular biological outcome. For example, in some circumstances, intrinsic activity may vary depending on the particular second messenger system involved. See Hoyer, D. and Boddeke, H., Trends Pharmacol. Sci. 14(7): 270-5 (1993), herein incorporated by reference with regard to such teaching.

As used herein, neurotransmitters whose release is mediated by the compounds described herein include, but are not limited to, acetylcholine, dopamine, norepinephrine, serotonin, and glutamate, and the compounds described herein function as modulators at the α7 subtype of the CNS NNRs.

As used herein, the terms “prevention” or “prophylaxis” include any degree of reducing the progression of or delaying the onset of a disease, disorder, or condition. The term includes providing protective effects against a particular disease, disorder, or condition as well as amelioration of the recurrence of the disease, disorder, or condition. Thus, in another aspect, the invention provides a method for treating a subject having or at risk of developing or experiencing a recurrence of a NNR or nAChR mediated disorder. The compounds and pharmaceutical compositions of the invention may be used to achieve a beneficial therapeutic or prophylactic effect, for example, in a subject with a CNS dysfunction.

As noted above, the free base and salt compounds of the present invention modulate the α7 NNR subtype, characteristic of the CNS, and can be used for preventing or treating various conditions or disorders, including those of the CNS, in subjects which have or are susceptible to such conditions or disorders, by modulation of the α7 NNR. The compounds have the ability to selectively bind to the α7 NNR and express nicotinic pharmacology. For example, compounds of the present invention, when administered in effective amounts to patients in need thereof, provide some degree of prevention of the progression of the CNS disorder, namely, providing protective effects, amelioration of the symptoms of the CNS disorder, or amelioration of the reoccurrence of the CNS disorder, or a combination thereof.

The compounds of the present invention can be used to treat or prevent those types of conditions and disorders for which other types of nicotinic compounds have been proposed or are shown to be useful as therapeutics. See, for example, the references previously listed hereinabove, as well as Williams et al., Drug News Perspec. 7(4): 205 (1994), Arneric et al., CNS Drug Rev. 1(1): 1-26 (1995), Arneric et al., Exp. Opin. Invest. Drugs 5(1): 79-100 (1996), Bencherif et al., J. Pharmacol. Exp. Ther. 279: 1413 (1996), Lippiello et al., J. Pharmacol. Exp. Ther. 279: 1422 (1996), Damaj et al., J. Pharmacol. Exp. Ther. 291: 390 (1999); Chiari et al., Anesthesiology 91: 1447 (1999), Lavand'homme and Eisenbach, Anesthesiology 91: 1455 (1999), Holladay et al., J. Med. Chem. 40(28): 4169-94 (1997), Bannon et al., Science 279: 77 (1998), PCT WO 94/08992, PCT WO 96/31475, PCT WO 96/40682, and U.S. Pat. Nos. 5,583,140 to Bencherif et al., 5,597,919 to Dull et al., 5,604,231 to Smith et al. and 5,852,041 to Cosford et al., the disclosures of which are incorporated herein by reference with regard to such therapeutic teaching.

The compounds and their pharmaceutical compositions are useful in the treatment or prevention of a variety of CNS disorders, including cognitive deficits and dysfunctions, age-related and otherwise and attentional disorders and, in particular schizophrenia.

As noted, schizophrenia can be divided into three major phases: the prodromal state, an active phase, and a residual phase. These phases tend to occur in sequence and appear in cycles throughout the course of the illness. The residual phase is similar to the prodromal phase although during the residual phase blunted affect and impairment in role functioning are more common. While psychotic symptoms may persist into the residual phase, the psychotic symptoms are less likely to be accompanied by such strong affect as experienced during the active phase. There is great variation in the severity of the residual phase from one person to the next. Some individuals will function extremely well while others may be considerably more impaired. After an active phase, people may be listless, have trouble concentrating and be withdrawn. The symptoms in this phase are similar to those outlined under the prodromal phase. If there have been no symptoms before the first episode, few or no symptoms may be experienced afterward. During a lifetime, people with schizophrenia may become actively ill once or twice, or have many more episodes. Unfortunately, residual symptoms may increase, while ability to function normally may decrease, after each active phase. It is therefore important to try to avoid relapses by following the prescribed treatment. Currently it is difficult to predict at the onset how fully a person will recover. The most common course of the disorder generally involves numerous active phases of illness with residual phases of impairment between episodes. The extent of residual impairment often increases between episodes during the initial years of the disorder although may possibly become less severe during the later phases of the illness.

Reference is made to Marder et al., Methodological Issues in Negative Symptom Trials, Schizophrenia Bulletin, 2011; and Laughren and Levin, Food and Drug Administration Commentary of Methodological Issues in Negative Symptom Trials, Schizophrenia Bulletin, 2011, each incorporated herein with regard to such teaching.

The above conditions and disorders are discussed in further detail, for example, in the American Psychiatric Association: Diagnostic and Statistical Manual of Mental Disorders, Fourth Edition, Text Revision, Washington, D.C., American Psychiatric Association, 2000; incorporated herein by reference with regard to defining such conditions and disorders. This Manual may also be referred to for greater detail on the symptoms and diagnostic features.

Preferably, the treatment or prevention of diseases, disorders, and conditions occurs without appreciable adverse side effects, including, for example, significant increases in blood pressure and heart rate, significant negative effects upon the gastro-intestinal tract, and significant effects upon skeletal muscle.

The compounds of the present invention, when employed in effective amounts, are believed to modulate the activity of the α7 NNR without appreciable interaction with the nicotinic subtypes that characterize the human ganglia, as demonstrated by a lack of the ability to elicit nicotinic function in adrenal chromaffin tissue, or skeletal muscle, further demonstrated by a lack of the ability to elicit nicotinic function in cell preparations expressing muscle-type nicotinic receptors. Thus, these compounds are believed capable of treating or preventing diseases, disorders, and conditions without eliciting significant side effects associated activity at ganglionic and neuromuscular sites. Thus, administration of the compounds is believed to provide a therapeutic window in which treatment of certain diseases, disorders, and conditions is provided, and certain side effects are avoided. That is, an effective dose of the compound is believed sufficient to provide the desired effects upon the disease, disorder, or condition, but is believed insufficient, namely is not at a high enough level, to provide undesirable side effects.

Thus, the present invention provides the use of a compound of the present invention, or a pharmaceutically acceptable salt thereof, for use in therapy, such as a therapy described above.

### V. Synthetic Examples

The following synthetic examples are provided to illustrate the present invention and should not be construed as limiting the scope thereof. In these examples, all parts and percentages are by weight, unless otherwise noted. All solutions are aqueous unless otherwise noted.

### Example 1

### Small Scale Synthesis of (2S,3R)—N-(2-((3-pyridinyl)methyl)-1-azabicyclo[2.2.2]octan-3-yl)benzofuran-2-carboxamide (Compound A) and its Enantiomer, (2R,3S)—N-(2-((3-pyridinyl)methyl)-1-azabicyclo[2.2.2]octan-3-yl)benzofuran-2-carboxamide

### 2-((3-Pyridinyl)methylene)-1-azabicyclo[2.2.2]octan-3-one

Potassium hydroxide (56 g, 0.54 mole) was dissolved in methanol (420 mL). 3-Quinuclidinone hydrochloride (75 g, 0.49 mole) was added and the mixture was stirred for 30 min at ambient temperature. 3-Pyridinecarboxaldehyde (58 g, 0.54 mole) was added and the mixture stirred for 16 h at ambient temperature. The reaction mixture became yellow during this period, with solids caking on the walls of the flask. The solids were scraped from the walls and the chunks broken up. With rapid stirring, water (390 mL) was added. When the solids dissolved, the mixture was cooled at 4° C. overnight. The crystals were collected by filtration, washed with water, and air dried to obtain 80 g of yellow solid. A second crop (8 g) was obtained by concentration of the filtrate to ˜10% of its former volume and cooling at 4° C. overnight. Both crops were sufficiently pure for further transformation (88 g, 82% yield).

### 2-((3-Pyridinyl)methyl)-1-azabicyclo[2.2.2]octan-3-one

2-((3-Pyridinyl)methylene)-1-azabicyclo[2.2.2]octan-3-one (20 g, 93 mmol) was suspended in methanol (200 mL) and treated with 46 mL of 6 M hydrochloric acid. 10% Palladium on carbon (1.6 g) was added and the mixture was shaken under 25 psi hydrogen for 16 h. The mixture was filtered through diatomaceous earth, and the solvent was removed from the filtrate by rotary evaporation. This provided crude 2-((3-pyridinyl)methyl)-1-azabicyclo[2.2.2]octan-3-one-hydrochloride, as a white gum (20 g), which was subsequently treated with 2 M sodium hydroxide (50 mL) and chloroform (50 mL) and stirred for an hour. The chloroform layer was separated, and the aqueous phase was treated with 2 M sodium hydroxide (˜5 mL, enough to raise the pH to 10) and saturated aqueous Sodium chloride (25 mL). This aqueous mixture was extracted with chloroform (3×10 mL), and the combined chloroform extracts were dried (anhydrous magnesium sulfate) and concentrated by rotary evaporation. The residue (18 g) was dissolved in warm ether (320 mL) and cooled to 4° C. The white solid was filtered off, washed with a small portion of cold ether and air dried. Concentration of the filtrate to ˜10% of its former volume and cooling at 4° C. produced a second crop. A combined yield 16 g (79%) was obtained.

### 3-Amino-2-((3-pyridinyl)methyl)-1-azabicyclo[2.2.2]octane

To a stirred solution of 2-((3-pyridinyl)methyl)-1-azabicyclo[2.2.2]octan-3-one (3.00 g, 13.9 mmol) in dry methanol (20 mL), under nitrogen, was added a 1 M solution of zinc chloride in ether (2.78 mL, 2.78 mmol). After stirring at ambient temperature for 30 min, this mixture was treated with solid ammonium formate (10.4 g, 167 mmol). After stirring another hour at ambient temperature, solid sodium cyanoborohydride (1.75 g, 27.8 mmol) was added in portions. The reaction was then stirred at ambient temperature overnight and terminated by addition of water (˜5 mL). The quenched reaction was partitioned between 5 M sodium hydroxide (10 mL) and chloroform (20 mL). The aqueous layer was extracted with chloroform (20 mL), and combined organic layers were dried (sodium sulfate), filtered and concentrated. This left 2.97 g of yellow gum. GCMS analysis indicated that the product was a 1:9 mixture of the cis and trans amines, along with a trace of the corresponding alcohol (98% total mass recovery).

### (2R,3S) and (2S,3R)-3-amino-2-((3-pyridinyl)methyl)-1-azabicyclo[2.2.2]octane

Di-p-toluoyl-D-tartaric acid (5.33 g, 13.8 mmol) was added to a stirred solution of crude 3-amino-2-((3-pyridinyl)methyl)-1-azabicyclo[2.2.2]octane (6.00 g, 27.6 mmol of 1:9 cis/trans) in methanol (20 mL). After complete dissolution, the clear solution was then concentrated to a solid mass by rotary evaporation. The solid was dissolved in a minimum amount of boiling methanol (˜5 mL). The solution was cooled slowly, first to ambient temperature (1 h), then for ˜4 h at 5° C. and finally at −5° C. overnight. The precipitated salt was collected by suction filtration and recrystallized from 5 mL of methanol. Air drying left 1.4 g of white solid, which was partitioned between chloroform (5 mL) and 2 M sodium hydroxide (5 mL). The chloroform layer and a 5 mL chloroform extract of the aqueous layer were combined, dried (anhydrous sodium sulfate) and concentrated to give a colorless oil (0.434 g). The enantiomeric purity of this free base was determined by conversion of a portion into its N-(tert-butoxycarbonyl)-L-prolinamide, which was then analyzed for diastereomeric purity (98%) using LCMS.

The mother liquor from the initial crystallization was made basic (˜pH 11) with 2 M sodium hydroxide and extracted twice with chloroform (10 mL). The chloroform extracts were dried (anhydrous sodium sulfate) and concentrated to give an oil. This amine (3.00 g, 13.8 mmol) was dissolved in methanol (10 mL) and treated with di-p-toluoyl-L-tartaric acid (2.76 g, 6.90 mmol). The mixture was warmed to aid dissolution and then cooled slowly to −5° C., where it remained overnight. The precipitate was collected by suction filtration, recrystallized from methanol and dried. This left 1.05 g of white solid. The salt was converted into the free base (yield=0.364 g), and the enantiomeric purity (97%) was assessed using the prolinamide method, as described above for the other enantiomer.

### Trans Enantiomer A of N-(2-((3-pyridinyl)methyl)-1-azabicyclo[2.2.2]octan-3-yl)benzofuran-2-carboxamide

Diphenylchlorophosphate (0.35 mL, 0.46 g, 1.7 mmol) was added drop-wise to a solution of benzofuran-2-carboxylic acid (0.28 g, 1.7 mmol) and triethylamine (0.24 mL, 0.17 g, 1.7 mmol) in dry dichloromethane (5 mL). After stirring at ambient temperature for 30 min, a solution of (2S,3R)-3-amino-2-((3-pyridinyl)methyl)-1-azabicyclo[2.2.2]octane (0.337 g, 1.55 mmol) (that derived from the di-p-toluoyl-D-tartaric acid salt) and triethylamine (0.24 mL, 0.17 g, 1.7 mmol) in dry dichloromethane (5 mL) was added. The reaction mixture was stirred overnight at ambient temperature, and then treated with 10% sodium hydroxide (1 mL). The biphasic mixture was separated, and the organic layer was concentrated on a Genevac centrifugal evaporator. The residue was dissolved in methanol (6 mL) and purified by HPLC on a C18 silica gel column, using an acetonitrile/water gradient, containing 0.05% trifluoroacetic acid, as eluent. Concentration of selected fractions, partitioning of the resulting residue between chloroform and saturated aqueous sodium bicarbonate, and evaporation of the chloroform gave 0.310 g (42% yield) of white powder (95% pure by GCMS). ¹H NMR (300 MHz, CDCl₃) δ 8.51 (d, 1H), 8.34 (dd, 1H), 7.66 (d, 1H), 7.58 (dt, 1H), 7.49 (d, 1H), 7.44 (s, 1H), 7.40 (dd, 1H), 7.29 (t, 1H), 7.13 (dd, 1H), 6.63 (d, 1H), 3.95 (t, 1H), 3.08 (m, 1H), 2.95 (m, 4H), 2.78 (m, 2H), 2.03 (m, 1H), 1.72 (m, 3H), 1.52 (m, 1H).

This material (trans enantiomer A) was later determined to be identical, by chiral chromatographic analysis, to material whose absolute configuration is 2S,3R (established by x-ray crystallographic analysis).

### Trans Enantiomer B of N-(2-((3-pyridinyl)methyl)-1-azabicyclo[2.2.2]octan-3-yl)benzofuran-2-carboxamide

Diphenylchlorophosphate (96 μL, 124 mg, 0.46 mmol) was added drop-wise to a solution of the benzofuran-2-carboxylic acid (75 mg, 0.46 mmol) and triethylamine (64 μL, 46 mg, 0.46 mmol) in dry dichloromethane (1 mL). After stirring at ambient temperature for 45 min, a solution of (2R,3S)-3-amino-2-((3-pyridinyl)methyl)-1-azabicyclo[2.2.2]octane (0.10 g, 0.46 mmol) (that derived from the di-p-toluoyl-L-tartaric acid salt) and triethylamine (64 μL, 46 mg, 0.46 mmol) in dry dichloromethane (1 mL) was added. The reaction mixture was stirred overnight at ambient temperature, and then treated with 10% sodium hydroxide (1 mL). The biphasic mixture was separated, and the organic layer and a chloroform extract (2 mL) of the aqueous layer was concentrated by rotary evaporation. The residue was dissolved in methanol and purified by HPLC on a C18 silica gel column, using an acetonitrile/water gradient, containing 0.05% trifluoroacetic acid, as eluent. Concentration of selected fractions, partitioning of the resulting residue between chloroform and saturated aqueous sodium bicarbonate, and evaporation of the chloroform gave 82.5 mg (50% yield) of a white powder. The NMR spectrum was identical to that obtained for the 2S,3R isomer. Since the immediate precursor of this material (trans enantiomer B) is enantiomeric to the immediate precursor of 2S,3R compound (trans enantiomer A), the absolute configuration of trans enantiomer B is presumed to be 2R,3S.

### Example 2

### Large Scale Synthesis of (2S,3R)—N-(2-((3-pyridinyl)methyl)-1-azabicyclo[2.2.2]octan-3-yl)benzofuran-2-carboxamide and (2S,3R)—N-(2-((3-pyridinyl)methyl)-1-azabicyclo[2.2.2]oct-3-yl)-1-benzofuran-2-carboxamide p-toluenesulfonate Salt

### 2-((3-Pyridinyl)methylene)-1-azabicyclo[2.2.2]octan-3-one

3-Quinuclidinone hydrochloride (8.25 kg, 51.0 mol) and methanol (49.5 L) were added to a 100 L glass reaction flask, under an nitrogen atmosphere, equipped with a mechanical stirrer, temperature probe, and condenser. Potassium hydroxide (5.55 kg, 99.0 mol) was added via a powder funnel over an approximately 30 min period, resulting in a rise in reaction temperature from 50° C. to 56° C. Over an approximately 2 h period, 3-pyridinecarboxaldehyde (4.80 kg, 44.9 mol) was added to the reaction mixture. The resulting mixture was stirred at 20° C.±5° C. for a minimum of 12 h, as the reaction was monitored by thin layer chromatography (TLC). Upon completion of the reaction, the reaction mixture was filtered through a sintered glass funnel and the filter cake was washed with methanol (74.2 L). The filtrate was concentrated, transferred to a reaction flask, and water (66.0 L) was added. The suspension was stirred for a minimum of 30 min, filtered, and the filter cake was washed with water (90.0 L) until the pH of the rinse was 7-9. The solid was dried under vacuum at 50° C.±5° C. for a minimum of 12 h to give 8.58 kg (89.3%) of 2-((3-pyridinyl)methylene)-1-azabicyclo[2.2.2]octan-3-one.

### (2S)-2-((3-pyridinyl)methyl)-1-azabicyclo[2.2.2]octan-3-one di-p-toluoyl-D-tartrate Salt

2-((3-Pyridinyl)methylene)-1-azabicyclo[2.2.2]octan-3-one (5.40 kg, 25.2 mol) and methanol (40.5 L) were added to a 72 L reaction vessel under an inert atmosphere equipped with a mechanical stirrer, temperature probe, low-pressure gas regulator system, and pressure gauge. The headspace was filled with nitrogen, and the mixture was stirred to obtain a clear yellow solution. To the flask was added 10% palladium on carbon (50% wet) (270 g). The atmosphere of the reactor was evacuated using a vacuum pump, and the headspace was replaced with hydrogen to 10 to 20 inches water pressure. The evacuation and pressurization with hydrogen were repeated 2 more times, leaving the reactor under 20 inches water pressure of hydrogen gas after the third pressurization. The reaction mixture was stirred at 20° C.±5° C. for a minimum of 12 h, and the reaction was monitored via TLC. Upon completion of the reaction, the suspension was filtered through a bed of Celite®545 (1.9 kg) on a sintered glass funnel, and the filter cake was washed with methanol (10.1 L). The filtrate was concentrated to obtain a semi-solid which was transferred, under an nitrogen atmosphere, to a 200 L reaction flask fitted with a mechanical stirrer, condenser, and temperature probe. The semi-solid was dissolved in ethanol (57.2 L), and di-p-toluoyl-D-tartaric acid (DTTA) (9.74 kg, 25.2 mol) was added. The stirring reaction mixture was heated at reflux for a minimum of 1 h, and for an additional minimum of 12 h while the reaction was cooled to between 15° C. and 30° C. The suspension was filtered using a tabletop filter, and the filter cake was washed with ethanol (11.4 L). The product was dried under vacuum at ambient temperature to obtain 11.6 kg (76.2% yield, 59.5% factored for purity) of (2S)-2-((3-pyridinyl)methyl)-1-azabicyclo[2.2.2]octan-3-one di-p-toluoyl-D-tartrate salt.

### (2S,3R)-3-Amino-2-((3-pyridinyl)methyl)-1-azabicyclo[2.2.2]octane di-p-toluoyl-D-tartrate Salt

Water (46.25 L) and sodium bicarbonate (4.35 kg, 51.8 mol) were added to a 200 L flask. Upon complete dissolution, dichloromethane (69.4 L) was added. (2S)-2-((3-Pyridinyl)methyl)-1-azabicyclo[2.2.2]octan-3-one di-p-toluoyl-D-tartrate salt (11.56 kg, 19.19 mol) was added, and the reaction mixture was stirred for between 2 min and 10 min. The layers were allowed to separate for a minimum of 2 min (additional water (20 L) was added when necessary to partition the layers). The organic phase was removed and dried over anhydrous sodium sulfate. Dichloromethane (34.7 L) was added to the remaining aqueous phase, and the suspension was stirred for between 2 min and 10 min. The layers were allowed to separate for between 2 min and 10 min. Again, the organic phase was removed and dried over anhydrous sodium sulfate. The extraction of the aqueous phase with dichloromethane (34.7 L) was repeated one more time, as above. Samples of each extraction were submitted for chiral HPLC analysis. The sodium sulfate was removed by filtration, and the filtrates were concentrated to obtain (2S)-2-((3-pyridinyl)methyl)-1-azabicyclo[2.2.2]octan-3-one (4.0 kg) as a solid.

The (2S)-2-((3-pyridinyl)methyl)-1-azabicyclo[2.2.2]octan-3-one (3.8 kg) was transferred to a clean 100 L glass reaction flask, under a nitrogen atmosphere, fitted with a mechanical stirrer and temperature probe. Anhydrous tetrahydrofuran (7.24 L) and (+)-(R)-α-methylbenzylamine (2.55 L, 20.1 mol) were added. Titanium(IV) isopropoxide (6.47 L, 21.8 mol) was added to the stirred reaction mixture over a 1 h period. The reaction was stirred under a nitrogen atmosphere for a minimum of 12 h. Ethanol (36.17 L) was added to the reaction mixture. The reaction mixture was cooled to below −5° C., and sodium borohydride (1.53 kg, 40.5 mol) was added in portions, keeping the reaction temperature below 15° C. (this addition took several hours). The reaction mixture was then stirred at 15° C.±10° C. for a minimum of 1 h. The reaction was monitored by HPLC, and upon completion of the reaction (as indicated by less than 0.5% of (2S)-2-((3-pyridinyl)methyl)-1-azabicyclo[2.2.2]octan-3-one remaining), 2 M sodium hydroxide (15.99 L) was added and the mixture was stirred for a minimum of 10 min. The reaction mixture was filtered through a bed of Celite®545 in a tabletop funnel. The filter cake was washed with ethanol (15.23 L), and the filtrate was concentrated to obtain an oil.

The concentrate was transferred to a clean 100 L glass reaction flask equipped with a mechanical stirrer and temperature probe under an inert atmosphere. Water (1 L) was added, and the mixture was cooled to 0° C.±5° C. 2 M Hydrochloric acid (24 L) was added to the mixture to adjust the pH of the mixture to pH 1. The mixture was then stirred for a minimum of 10 min, and 2 M sodium hydroxide (24 L) was slowly added to adjust the pH of the mixture to pH 14. The mixture was stirred for a minimum of 10 min, and the aqueous phase was extracted with dichloromethane (3×15.23 L). The organic phases were dried over anhydrous sodium sulfate (2.0 kg), filtered, and concentrated to give (2S,3R)—N-((1R)-phenylethyl)-3-amino-2-((3-pyridinyl)methyl))-1-azabicyclo[2.2.2]octane (4.80 kg, 84.7% yield).

The (2S,3R)—N-((1R)-phenylethyl)-3-amino-2-((3-pyridinyl)methyl)-1-azabicyclo[2.2.2]octane was transferred to a 22 L glass flask equipped with a mechanical stirrer and temperature probe under an inert atmosphere. Water (4.8 L) was added, and the stirring mixture was cooled to 5° C.±5° C. Concentrated hydrochloric acid (2.97 L) was slowly added to the reaction flask, keeping the temperature of the mixture below 25° C. The resulting solution was transferred to a 72 L reaction flask containing ethanol (18 L), equipped with a mechanical stirrer, temperature probe, and condenser under an inert atmosphere. To the flask was added 10% palladium on carbon (50% wet) (311.1 g) and cyclohexene (14.36 L). The reaction mixture was heated at near-reflux for a minimum of 12 h, and the reaction was monitored by TLC. Upon completion of the reaction, the reaction mixture was cooled to below 45° C., and it was filtered through a bed of Celite®545 (1.2 kg) on a sintered glass funnel. The filter cake was rinsed with ethanol (3 L) and the filtrate was concentrated to obtain an aqueous phase. Water (500 mL) was added to the concentrated filtrate, and this combined aqueous layer was washed with methyl tert-butyl ether (MTBE) (2×4.79 L). 2 M Sodium hydroxide (19.5 L) was added to the aqueous phase to adjust the pH of the mixture to pH 14. The mixture was then stirred for a minimum of 10 min. The aqueous phase was extracted with chloroform (4×11.96 L), and the combined organic phases were dried over anhydrous sodium sulfate (2.34 kg). The filtrate was filtered and concentrated to obtain (2S,3R)-3-amino-2-((3-pyridinyl)methyl)-1-azabicyclo[2.2.2]octane (3.49 kg, >quantitative yield) as an oil.

The (2S,3R)-3-amino-2-((3-pyridinyl)methyl)-1-azabicyclo[2.2.2]octane was transferred to a clean 100 L reaction flask equipped with a mechanical stirrer, condenser, and temperature probe under an inert atmosphere. Ethanol (38.4 L) and di-p-toluoyl-D-tartaric acid (3.58 kg, 9.27 mol) were added. The reaction mixture was heated at gentle reflux for a minimum of 1 h. The reaction mixture was then stirred for a minimum of 12 h while it was cooled to between 15° C. and 30° C. The resulting suspension was filtered, and the filter cake was washed with ethanol (5.76 L). The filter cake was transferred to a clean 100 L glass reaction flask equipped with a mechanical stirrer, temperature probe, and condenser under an inert atmosphere. A 9:1 ethanol/water solution (30.7 L) was added, and the resulting slurry was heated at gentle reflux for a minimum of 1 h. The reaction mixture was then stirred for a minimum of 12 h while cooling to between 15° C. and 30° C. The mixture was filtered and the filter cake was washed with ethanol (5.76 L). The product was collected and dried under vacuum at 50° C.±5° C. for a minimum of 12 h to give 5.63 kg (58.1% yield) of (2S,3R)-3-amino-2-((3-pyridinyl)methyl)-1-azabicyclo[2.2.2]octane di-p-toluoyl-D-tartrate salt.

### (2S,3R)—N-(2-((3-Pyridinyl)methyl)-1-azabicyclo[2.2.2]octan-3-yl)benzofuran-2-carboxamide

(2S,3R)-3-Amino-2-((3-pyridinyl)methyl)-1-azabicyclo[2.2.2]octane di-p-toluoyl-D-tartrate salt (3.64 kg, 5.96 mol) and 10% aqueous sodium chloride solution (14.4 L, 46.4 mol) were added to a 72 L glass reaction flask equipped with a mechanical stirrer under an inert atmosphere. 5 M Sodium hydroxide (5.09 L) was added to the stirring mixture to adjust the pH of the mixture to pH 14. The mixture was then stirred for a minimum of 10 min. The aqueous solution was extracted with chloroform (4×12.0 L), and the combined organic layers were dried over anhydrous sodium sulfate (1.72 kg). The combined organic layers were filtered, and the filtrate was concentrated to obtain (2S,3R)-3-amino-2-((3-pyridinyl)methyl)-1-azabicyclo[2.2.2]octane (1.27 kg) as an oil.

The (2S,3R)-3-amino-2-((3-pyridinyl)methyl)-1-azabicyclo[2.2.2]octane was transferred to a 50 L glass reaction flask equipped with a mechanical stirrer under an inert atmosphere. Dichloromethane (16.5 L), triethylamine (847 mL, 6.08 mol), benzofuran-2-carboxylic acid (948 g, 5.85 mol) and O-(benzotriazol-1-yl)-N,N,N,1-tetramethyluronium hexafluorophosphate (HBTU) (2.17 kg, 5.85 mol) were added to the reaction mixture. The mixture was stirred for a minimum of 4 h at ambient temperature, and the reaction was monitored by HPLC. Upon completion of the reaction, 10% aqueous potassium carbonate (12.7 L, 17.1 mol) was added to the reaction mixture and the mixture was stirred for a minimum of 5 min. The layers were separated and the organic phase was washed with 10% brine (12.7 L). The layers were separated and the organic phase was cooled to 15° C.±10° C. 3 M Hydrochloric acid (8.0 L) was slowly added to the reaction mixture to adjust the pH of the mixture to pH 1. The mixture was then stirred for a minimum of 5 min, and the layers were allowed to partition for a minimum of 5 min. The solids were filtered using a table top filter. The layers of the filtrate were separated, and the aqueous phase and the solids from the funnel were transferred to the reaction flask. 3 M Sodium hydroxide (9.0 L) was slowly added to the flask in portions to adjust the pH of the mixture to pH 14. The aqueous phase was extracted with dichloromethane (2×16.5 L). The combined organic phases were dried over anhydrous sodium sulfate (1.71 kg). The mixture was filtered, and the filtrate was concentrated to give (2S,3R)—N-(2-((3-pyridinyl)methyl)-1-azabicyclo[2.2.2]octan-3-yl)benzofuran-2-carboxamide (1.63 kg, 77.0% yield) as a yellow solid.

### (2S,3R)—N-(2-((3-pyridinyl)methyl)-1-azabicyclo[2.2.2]oct-3-yl]benzofuran-2-carboxamide p-toluenesulfonate

(2S,3R)—N-(2-((3-Pyridinyl)methyl)-1-azabicyclo[2.2.2]octan-3-yl)benzofuran-2-carboxamide (1.62 kg, 4.48 mol) and dichloromethane (8.60 kg) were added into a carboy. The weight/weight percent of the material in solution was determined through HPLC analysis. The solution was concentrated to an oil, acetone (4 L) was added, and the mixture was concentrated to an oily solid. Additional acetone (12 L) was added to the oily solid in the rotary evaporator bulb, and the resulting slurry was transferred to a 50 L glass reaction flask with a mechanical stirrer, condenser, temperature probe, and condenser under an inert atmosphere. The reaction mixture was heated to 50° C.±5° C. Water (80.7 g) was added to the solution, and it was stirred for a minimum of 10 min. p-Toluenesulfonic acid (853 g, 4.44 mol) was added to the reaction mixture in portions over approximately 15 min. The reaction mixture was heated to reflux and held at that temperature for a minimum of 30 min to obtain a solution. The reaction was cooled to 40° C.±5° C. over approximately 2 h. Isopropyl acetate (14.1 L) was added over approximately 1.5 h. The reaction mixture was slowly cooled to ambient temperature over a minimum of 10 h. The mixture was filtered and the filter cake was washed with isopropyl acetate (3.5 L). The isolated product was dried under vacuum at 105° C.±5° C. for between 2 h and 9 h to give 2.19 kg (88.5% yield) of (2S,3R)—N-(2-((3-pyridinyl)methyl)-1-azabicyclo[2.2.2]oct-3-yl)benzofuran-2-carboxamide p-toluenesulfonate, mp 226-228° C. ¹H NMR (500 MHz, D₂O) δ 8.29 (s, 1H), 7.78 (m, J=5.1, 1H), 7.63 (d, J=7.9, 1H), 7.54 (d, J=7.8, 1H), 7.49 (d, J=8.1, 2H), 7.37 (m, J=8.3, 1H), 7.33 (m, J=8.3, 6.9, 1.0, 1H), 7.18 (m, J=7.8, 6.9, 1.0, 1H), 7.14 (d, J=8.1, 2H), 7.09 (s, 1H), 6.99 (dd, J=7.9, 5.1, 1H), 4.05 (m, J=7.7, 1H), 3.74 (m, 1H), 3.47 (m, 2H), 3.28 (m, 1H), 3.22 (m, 1H), 3.15 (dd, J=13.2, 4.7, 1H), 3.02 (dd, J=13.2, 11.5, 1H), 2.19 (s, 3H), 2.02 (m, 2H), 1.93 (m, 2H), 1.79 (m, 1H). ¹³C NMR (126 MHz, D₂O) δ 157.2, 154.1, 150.1, 148.2, 146.4, 145.2, 138.0, 137.0, 130.9, 128.2 (2), 126.9, 126.8, 125.5 (2), 123.7, 123.3, 122.7, 111.7, 100.7, 61.3, 50.2, 48.0, 40.9, 33.1, 26.9, 21.5, 20.8, 17.0.

Samples of this material were converted into Compound A free base (for use in salt selection studies) by treatment with aqueous sodium hydroxide and extraction with chloroform. Thorough evaporation of the chloroform left an off-white powder, mp 167-170° C., with the following spectral characteristics: Positive ion electrospray MS [M+H]⁺ ion m/z=362. ¹H NMR (500 MHz, DMSO-d₆) δ 8.53 (d, J=7.6 Hz, 1H), 8.43 (d, J=1.7 Hz, 1H), 8.28 (dd, J=1.6, 4.7 Hz, 1H), 7.77 (d, J=7.7 Hz, 1H), 7.66 (d, J=8.5 Hz, 1H), 7.63 (dt, J=1.7, 7.7 Hz, 1H), 7.52 (s, 1H), 7.46 (m, J=8.5, 7.5 Hz, 1H), 7.33 (m, J=7.7, 7.5 Hz, 1H), 7.21 (dd, J=4.7, 7.7 Hz, 1H), 3.71 (m, J=7.6 Hz, 1H), 3.11 (m, 1H), 3.02 (m, 1H), 2.80 (m, 2H), 2.69 (m, 2H), 2.55 (m, 1H), 1.80 (m, 1H), 1.77 (m, 1H), 1.62 (m, 1H), 1.56 (m, 1H), 1.26 (m, 1H). ¹³C NMR (126 MHz, DMSO-d₆) δ 158.1, 154.1, 150.1, 149.1, 146.8, 136.4, 135.4, 127.1, 126.7, 123.6, 122.9, 122.6, 111.8, 109.3, 61.9, 53.4, 49.9, 40.3, 35.0, 28.1, 26.1, 19.6.

The monohydrochloride salt of Compound A (see Example 3) was submitted for x-ray crystallographic analysis. The resulting crystal structure established the 2S,3R absolute configuration of Compound A.

### Example 3

### Synthesis of (2S,3R)—N-(2-((3-pyridinyl)methyl)-1-azabicyclo[2.2.2]oct-3-yl)benzofuran-2-carboxamide Hydrochloride Salts

Monohydrochloride: A hydrochloric acid/THF solution was prepared by adding of concentrated hydrochloric acid (1.93 mL of 12M, 23.2 mmol) drop-wise to 8.5 mL of chilled THF.

The solution was warmed to ambient temperature. To a round bottom flask was added (2S,3R)—N-(2-((3-pyridinyl)methyl)-1-azabicyclo[2.2.2]oct-3-yl)benzofuran-2-carboxamide (8.49 g, 23.5 mmol) and acetone (85 mL). The mixture was stirred and heated at 45-50° C. until a complete solution was obtained. The hydrochloric acid/THF solution prepared above was added drop-wise over a 5 min period, with additional THF (1.5 mL) used in the transfer. Granular, white solids began to form during the addition of the acid solution. The mixture was cooled to ambient temperature, and stirred overnight (16 h). The solids were collected by suction filtration, the filter cake was washed with acetone (10 mL), and the solid was air-dried with suction for 30 min. The solid was further dried in a vacuum oven at 75° C. for 2 h to give 8.79 g of the fine white crystals (94% yield), mp 255-262° C. Chiral LC analysis gave a purity of 98.8% (270 nm). ¹H-NMR (DMSO-d₆) shows no residual solvents and confirms mono stoichiometry. ¹H NMR (300 MHz, DMSO-d₆) δ 10.7 (broad s, 1H—quaternary ammonium), 8.80 (broad s, 1H—amide H), 8.54 (s, 1H), 8.23 (d, 1H), 7.78 (d, 1H), 7.74 (d, 1H), 7.60 (d, 1H), 7.47 (m, 2H), 7.33 (m, 1H), 7.19 (m, 1H), 4.19 (m, 1H), 4.08 (m, 1H), 3.05-3.55 (m, 6H), 2.00-2.10 (m, 3H), 1.90 (m, 1H), 1.70 (m, 1H). An x-ray crystallographic analysis of this salt established stereochemical assignment and stoichiometry.

Dihydrochloride: Hydrogen chloride gas was slowly bubbled into a ice cooled solution of (2S,3R)—N-(2-((3-pyridinyl)methyl)-1-azabicyclo[2.2.2]oct-3-yl)benzofuran-2-carboxamide (1.9 g, 5.3 mmol) in anhydrous ether (25 mL). The volatiles were removed, first in a nitrogen stream and then with high vacuum (sodium hydroxide scrubber in high vacuum line). The residue was triturated several times with small volumes of anhydrous ether (discarded), and the remaining solid was dried under high vacuum. This gave 2.17 g (94% yield) of off-white powder, mp 210-212° C. (hygroscopic). Chiral LC analysis gave a purity of 93.7% (270 nm). Positive ion electrospray MS [M+H]⁺ ion m/z=362. ¹H NMR (300 MHz, CD₃OD) δ 9.15 (s, 1H), 8.84 (d, 1H), 8.63 (d, 1H), 7.97 (t, 1H), 7.75 (d, 1H), 7.61 (d, 1H), 7.52 (m, 2H), 7.35 (t, 1H), 4.50 (m, 1H), 4.32 (m, 1H), 3.40-3.85 (m, 6H), 1.95-2.40 (m, 5H).

### VI. A Double-Blind, Placebo-Controlled, Multicenter, Parallel Group Study to Assess Efficacy, Safety, and Tolerability of (2S,3R)—N-(2-((3-pyridinyl)methyl)-1-azabicyclo[2.2.2]oct-3-yl)benzofuran-2-carboxamide or a Pharmaceutically Acceptable Salt Thereof as Augmentation Therapy to Improve Cognition in Outpatients with Cognitive Dysfunction in Schizophrenia

A Phase 2 clinical proof of concept trial was conducted to evaluate (2S,3R)—N-(2-((3-pyridinyl)methyl)-1-azabicyclo[2.2.2]oct-3-yl)benzofuran-2-carboxamide pharmaceutically acceptable salt as an augmentation therapy to improve cognition in patients with schizophrenia. In the trial, (2S,3R)—N-(2-((3-pyridinyl)methyl)-1-azabicyclo[2.2.2]oct-3-yl)benzofuran-2-carboxamide or pharmaceutically acceptable salt thereof met the protocol criteria for a positive result on the primary efficacy outcome measure, the Groton Maze Learning Task (GMLT) of the CogState Schizophrenia Battery (CogState, New Haven, Conn.; http://www.cogstate.com/go/clinicaltrials/our-test-batteries/schizophrenia-battery), and was well tolerated.

As provided in the above-referenced brochure, CogState is a provider of cognitive testing products and services that predominantly caters to the global pharmaceutical industry. The Schizophrenia Cognitive Test Battery covers all domains identified by MATRICS initiative; requires around 35 minutes for administration; is sensitive to the effects of novel compounds and licensed medication; is sensitive to the effects of medication in groups or individual patients; and the outcome measures are related to functional status.

There is sensitivity of three measures from the CogState schizophrenia battery to change in cognitive function in schizophrenia. FIG. 1 summarizes the results from a placebo controlled parallel group study comparing the effect of a novel alpha 7 nicotine agonist on the Simple Reaction Time (information processing), International Shopping List (verbal learning) and Groton Maze Learning Task (problem solving) tasks in patients with chronic schizophrenia (n=30 per group) who were stable on their antipsychotic medication at randomization. Improvement in performance of greater than 0.4 standard deviation units was found for each measure.

FIG. 2 illustrates sensitivity of the primary outcome measures from the CogState schizophrenia battery to cognitive impairment in patients with chronic schizophrenia who were receiving antipsychotic medication in three different geographical areas. The nature and magnitude of impairment in the different cognitive domains was consistent across the three cultural groups.

As a result of FDA and NIH agreement that cognitive dysfunction/impairment is an important health issue in schizophrenia, the FDA confirmed that improved cognitive function would be considered as a primary outcome in schizophrenia research. Following this guidance, the MATRICS initiative, a collaboration of FDA, NIH, academic researchers, and industry, recommended a test battery for the assessment of cognition in this population. Prior to the MATRICS initiative, there was no consensus on the optimum outcome measures for evaluation of cognitive function in clinical trials in schizophrenia. The test criteria recommended by the MATRICS group was defined as:

High Test-Retest Reliability
Good coverage of cognitive domains
Comparable alternative forms
Strong internal consistency
Well established in general population
Demonstrated tolerability and acceptability

All CogState tasks meet the criteria set out above with regards to scientific validity, reliability and consistency. These tasks, when put together to form the CogState Schizophrenia Battery, also cover the cognitive domains stipulated by the MATRICS initiative.

The test battery is rapid. It covers all necessary domains in around 35 minutes—a benefit when working with patients suffering from schizophrenia. The user-friendly test battery allows for non-expert administration on standard computer equipment, which reduces costs and increases efficiency. The tasks utilize culture-neutral stimuli, which ensures that they can be integrated into clinical trials all around the world regardless of culture, ethnicity and socio-economic status. The test battery has high test-retest reliability, which ensures quality data.

TABLE 1
The CogState Schizophrenia Battery

| Domain | CogState Task | Task Description |
|---|---|---|
| Speed of | Detection | The pre-task on-screen instructions ask: “Has the card |
| Processing/Simple | | turned over?” A playing card is presented in the center |
| Reaction Time | | of the screen. The card will flip over so it is face up. As |
| | | soon as it does, the subject must press the “Yes” key. |
| | | The card will go to the back of the pack and the |
| | | subject must press the “Yes” key as soon as the next |
| | | card flips over and so on. |
| Attention/Vigilance | Identification | The pre-task on-screen instructions ask: “Is the card |
| | | red?” A playing card is presented in the center of the |
| | | screen. The card will flip over so it is face up. As soon |
| | | as it does this the subject must decide whether the |
| | | card is red or not. If it is red they should press “Yes”, if |
| | | it is not red they should press “No”. |
| Working Memory | One-Back | The pre-task on-screen instructions ask: “Does the |
| | | face up card exactly match the one before?” A playing |
| | | card is presented face up in the center of the screen. |
| | | The subject must decide as each card is presented |
| | | whether it is identical to the one just before. If the card |
| | | is identical to the card presented immediately before it, |
| | | they should press “Yes” if it is not they should press |
| | | “No”. |
| Visual Learning | One Card Learning | The pre-task on-screen instructions ask: “Have you |
| | | seen this card before in this task?” A playing card is |
| | | presented in the center of the screen. As soon as it |
| | | does the subject must decide whether or not the same |
| | | card has been seen before in this task. Subjects must |
| | | try to remember all the cards that have been shown |
| | | previously in order to decide whether or not they have |
| | | seen each card before. |
| Verbal Learning | International | The subject is told by the test supervisor: “In this task, |
| | Shopping List | I am going to read you a shopping list. I would like you |
| | | to remember as many items from this list as possible. |
| | | Are you ready to start?” The test supervisor reads the |
| | | list of words as they appear on the computer screen. |
| | | When the test supervisor has read all the words, the |
| | | subject is required to recall as many items as possible |
| | | from the list. |
| Reasoning/Problem | Groton Maze | The subject is shown a 10 × 10 grid of tiles on a |
| Solving | Learning | computer touch screen. A 28-step pathway is hidden |
| | | among these 100 possible locations. The start is |
| | | indicated by a blue tile at the top left and the finish |
| | | location is a tile with the red circles at the bottom right |
| | | of the grid. The subject is instructed to move one step |
| | | from the start location and then to continue, one tile at |
| | | a time, toward the end (bottom right). |
| Social Cognition | Social-Emotional | The pre-task on-screen instructions ask, “Tap the odd |
| | Cognition | one out”. In this task, the subject will see a number of |
| | | pictures on the screen. One of these pictures will be |
| | | different to the others in some way. The subject must |
| | | decide which one of the pictures is different, then tap |
| | | that picture as quickly as they can. |

The double blind, placebo controlled Phase 2 trial was conducted at 7 sites in the United States and 12 sites in India. In the trial, 185 patients meeting DSM-IV criteria for schizophrenia, with stable psychotic symptoms and taking a stable dose of an approved atypical antipsychotic medication (either quetiapine, marketed as Seroquel®, or risperidone, marketed as Risperdal®) were randomized to receive either (2S,3R)—N-(2-((3-pyridinyl)methyl)-1-azabicyclo[2.2.2]oct-3-yl)benzofuran-2-carboxamide pharmaceutically acceptable salt or placebo, together with continued treatment with the atypical antipsychotic, for 12 weeks. Of the randomized patients, approximately 69% were male and approximately 46% were users of tobacco products. Patients who received (2S,3R)—N-(2-((3-pyridinyl)methyl)-1-azabicyclo[2.2.2]oct-3-yl)benzofuran-2-carboxamide pharmaceutically acceptable salt received 1 mg daily dose for the first four weeks, a 5 mg daily dose for the next four weeks and a 25 mg daily dose for the last four weeks. The primary efficacy outcome measure was GMLT, and the trial included a number of other scales as secondary efficacy outcome measures.

The GMLT is a computerized test designed to assess executive function (the ability to organize cognitive processes, including the ability to plan, prioritize, stop and start activities, shift from one activity to another activity and to monitor one's own behavior). Impaired executive function is thought to be an important aspect of cognitive dysfunction in schizophrenia. The trial protocol (see hereinafter—the Protocol) defined a positive outcome on GMLT as superiority (one-sided p-value <0.10) for the (2S,3R)—N-(2-((3-pyridinyl)methyl)-1-azabicyclo[2.2.2]oct-3-yl)benzofuran-2-carboxamide pharmaceutically acceptable salt dose group as compared to the placebo dose group after adjusting statistically to account for 3 multiple comparisons (at Weeks 4, 8, and 12 to evaluate the 3 doses).

In the trial, the results on GMLT met the pre-defined success criteria (adjusted p-value=0.054), as well as at two of the trial's three measurement dates (at 4 weeks, unadjusted p-value=0.018; and at 12 weeks, unadjusted p-value=0.041); and were favorable for tobacco users as compared to non-users and for patients at study sites in United States as compared to patients at study sites in India. It has been estimated that approximately 80% of schizophrenic patients smoke (Swan& Lessoc-Schlaggar, Neuropsychological Review, 17:259-273, 2007); there was no activity in non-users. Each of the p-values reported was derived after data log transformation, a commonly utilized technique where the distribution of data is skewed (not normally distributed).

In addition, encouraging efficacy signals were observed in the trial on several secondary outcome measures, including Scale for Assessment of Negative Symptoms, an investigator assessment of improvement on the negative symptoms of schizophrenia, Clinical Global Impression—Global Improvement, an investigator assessment of overall response, and Subject Global Impression—Cognition scale, a patient self-assessment of cognitive change. Clinical Global Impression—Severity, did not demonstrate a drug effect. Further, while certain secondary outcome measures of the trial, including cognitive measures in the CogState Schizophrenia Test Battery, did not demonstrate a drug effect, several CogState objective cognitive endpoints, including but not limited to composite score, detection (psychomotor speed), identification (attention), 1-card learning (visual learning), 1-back (working memory), and International Shopping List (verbal learning), provided statistically significant results of improvement.

Table 2 provides the results of primary, secondary, and CogState analyses of the total population (tobacco users and non-users).

TABLE 2
Results of Primary, Secondary and CogState* analyses, total population (tobacco users and non-users)

| TASK | WEEK 4** | WEEK 8** | WEEK 12** |
|---|---|---|---|
| GMLT log (10) | 0.05 (0.02); p = 0.018 | 0.02 (0.02); p = 0.131 | 0.04 (0.02); p = 0.041 |
| transformed | | | |
| SANS | 0.78 (1.18); p = 0.255 | 1.71 (1.44); p = 0.118 | 3.70 (1.68); p = 0.015 |
| CGI-I | 0.15 (0.09); p = 0.049 | 0.08 (0.12); p = 0.253 | 0.15 (0.14); p = 0.150 |
| SGI-Cog | 0.05 (0.28); p = 0.428 | 0.01 (0.36); p = 0.491 | 0.66 (0.39); p = 0.046 |
| CGI-S | −0.02 (0.06); p = 0.674 | −0.01 (0.06); p = 0.544 | 0.01 (0.09); p = 0.450 |
| CogState | −0.066 (0.066); p = 0.838 | −0.062 .068); p = 0.821 | 0.030 0.067); p = 0.326 |
| Composite | | | |
| score | | | |
| CogState | 0.007 (0.015); p = 0.693 | 0.006 (0.014) p = 0.650 | 0.002 (0.015) p = 0.567 |
| Detection | | | |
| CogState | −0.005 (0.011); p = 0.311 | −0.014 (0.012); p = 0.111 | −0.017 (0.011); p = 0.063 |
| Identification | | | |
| CogState | −0.008 (0.019); P = 0.660 | −0.024 (0.019); P = 0.899 | −0.019 (0.020); P = 0.841 |
| 1-Card | | | |
| Learning | | | |
| CogState | 0.007 (0.014); p = 0.691 | 0.005 (0.015); p = 0.647 | −0.024 (0.014); p = 0.042 |
| 1-Back | | | |
| CogState | 0.013 (0.020); p = 0.259 | 0.015 (0.020); p = 0.217 | −0.002 (0.020); p = 0.546 |
| Social- | | | |
| Emotional | | | |
| Cognition | | | |

*Rows for CogState Composite score through Social-Emotional Cognition Task depict results in subjects meeting data integrity criteria.
**Values in each cell are: [Mean (SEM); 1-tailed p-value]

Table 3 provides the results of primary, secondary, and CogState analyses in tobacco users.

TABLE 3
Results of Primary, Secondary and CogState analyses* in tobacco users

| | Week 4** | Week 8** | Week 12** |
|---|---|---|---|
| GMLT log (10) | 0.06 (.03) p = 0.014 | 0.04 (0.03) p = 0.086 | 0.07 (0.02) p = 0.002 |
| transformed | | | |
| SANS | 2.74 (2.10); p = 0.098 | 4.00 (2.45); p = 0.054 | 4.86 (2.58); p = 0.033 |
| CGI-I | 0.25 (0.14); p = 0.047 | 0.24 (0.16); p = 0.075 | 0.18 (0.20); p = 0.189 |
| SGI-Cog | −0.13 (0.47); p = 0.606 | 0.24 (0.49); p = 0.314 | 0.65 (0.52); p = 0.109 |
| CGI-S | 0.02 (0.09); p = 0.434 | 0.08 (0.10); p = 0.224 | 0.07 (0.14); p = 0.308 |
| CogState | 0.007 0.099); p = 0.471 | −0.029 (0.101); p = 0.614 | 0.129 0.100); p = 0.098 |
| Composite | | | |
| score | | | |
| CogState | −0.014 (0.021); p = 0.254 | −0.000 (0.021); p = 0.491 | −0.013 0.021); p = 0.265 |
| Detection | | | |
| CogState | −0.021 (0.016); p = 0.097 | −0.011 (0.017); p = 0.261 | −0.026 (0.016); p = 0.057 |
| Identification | | | |
| CogState | −0.008 (0.027); p = 0.615 | −0.021; 0.028); p = 0.778 | 0.012 (0.028); p = 0.338 |
| 1-Card | | | |
| Learning | | | |
| CogState | 0.005 (0.021); p = 0.605 | −0.009 (0.021); p = 0.329 | −0.042 (0.020); p = 0.020 |
| 1-Back | | | |
| CogState | −0.010 (0.028); p = 0.632 | −0.009 (0.029); p = 0.625 | −0.019 (0.027); p = 0.752 |
| Social- | | | |
| Emotional | | | |
| Cognition | | | |

*All CogState results except for GMLT are in subjects meeting data integrity criteria. The methodology for GMLT does not use a data integrity criterion.
**Values in each cell are: [Mean (SE); 1-tailed p-value]

The statistically significant and qualitatively similar effects favoring (2S,3R)—N-(2-((3-pyridinyl)methyl)-1-azabicyclo[2.2.2]oct-3-yl)benzofuran-2-carboxamide pharmaceutically acceptable salt in the primary objective endpoint (GMLT), in a number of secondary clinician and patient-rated endpoints (SANS, CGI-Global, and SGI-Cog), and in CogState objective cognitive endpoints underscore the positive efficacy of (2S,3R)—N-(2-((3-pyridinyl)methyl)-1-azabicyclo[2.2.2]oct-3-yl)benzofuran-2-carboxamide or a pharmaceutically acceptable salt thereof in Cognitive Deficits in Schizophrenia.

(2S,3R)—N-(2-((3-pyridinyl)methyl)-1-azabicyclo[2.2.2]oct-3-yl)benzofuran-2-carboxamide or a pharmaceutically acceptable salt thereof exhibited a favorable tolerability profile in the trial, and there was no clinically significant difference between the (2S,3R)—N-(2-((3-pyridinyl)methyl)-1-azabicyclo[2.2.2]oct-3-yl)benzofuran-2-carboxamide or a pharmaceutically acceptable salt thereof and placebo dose groups in discontinuations due to adverse events. The most frequent adverse event that was more common in the (2S,3R)—N-(2-((3-pyridinyl)methyl)-1-azabicyclo[2.2.2]oct-3-yl)benzofuran-2-carboxamide pharmaceutically acceptable salt cohort than in the placebo cohort was nausea (0% placebo vs. 5% (2S,3R)—N-(2-((3-pyridinyl)methyl)-1-azabicyclo[2.2.2]oct-3-yl)benzofuran-2-carboxamide pharmaceutically acceptable salt), which was mild to moderate in severity and never led to patient dropout. There were two serious adverse events in the trial, one in the placebo dose group and one in the (2S,3R)—N-(2-((3-pyridinyl)methyl)-1-azabicyclo[2.2.2]oct-3-yl)benzofuran-2-carboxamide pharmaceutically acceptable salt dose group. Both were considered by the applicable investigator as not drug related.

The efficacy of (2S,3R)—N-(2-((3-pyridinyl)methyl)-1-azabicyclo[2.2.2]oct-3-yl)benzofuran-2-carboxamide or a pharmaceutically acceptable salt thereof against negative symptoms and cognitive symptoms is a remarkable finding due to the relative lack of effect of atypical antipsychotics upon these residual symptoms of schizophrenia. Because these residual symptoms are the primary reason that people with schizophrenia do not regain their full pre-morbid level of function, a new treatment for these symptoms fills a major unmet need. This need has been recognized by the NIMH through their MATRICS initiative (Neuchterlein et al., 2004; Gold, 2004), other initiatives with broad academic and regulatory support (Blanchard et al., 2010; Marder et al., 2011), and endorsed by the FDA (Laughren and Levin, 2011). The MATRICS initiative has highlighted the potential for small molecules that target the alpha7 NNR receptor in the treatment for cognitive dysfunction in schizophrenia. That potential was supported by preclinical models of schizophrenia in which the alpha 7 NNR agonist, (2S,3R)—N-(2-((3-pyridinyl)methyl)-1-azabicyclo[2.2.2]oct-3-yl)benzofuran-2-carboxamide or a pharmaceutically acceptable salt thereof, was effective; and by early clinical studies in which a variety of other alpha7 NNR agonists were effective against surrogate markers (Olincy et al., 2006; EnVivo Pharmaceuticals, 2009) and measured features of schizophrenia (Freedman et al., 2008).

(2S,3R)—N-(2-((3-pyridinyl)methyl)-1-azabicyclo[2.2.2]oct-3-yl)benzofuran-2-carboxamide or a pharmaceutically acceptable salt thereof showed efficacy primarily in tobacco users. Without being limited thereto, there are at least 3 hypotheses that may explain this observation. First, in schizophrenic patients who smoke, mRNA encoding the alpha7 NNR subunit was shown to be upregulated by 250% in comparison to schizophrenic patients who did not smoke; and there were a greater number of functional alpha7 NNR receptors in the smokers (Mexal et al., 2010). This finding may suggest that there were more functionally active alpha7 receptors upon which (2S,3R)—N-(2-((3-pyridinyl)methyl)-1-azabicyclo[2.2.2]oct-3-yl)benzofuran-2-carboxamide or a pharmaceutically acceptable salt thereof could act in the brains of the tobacco-users in this study, with a greater functional consequence. Second, nicotine has been demonstrated to make the blood-brain barrier more permeable to small molecules in preclinical models (Hawkins et al., 2004; Manda et al., 2010). If this effect of nicotine is also present in humans, then it is possible that a greater brain concentration of (2S,3R)—N-(2-((3-pyridinyl)methyl)-1-azabicyclo[2.2.2]oct-3-yl)benzofuran-2-carboxamide or a pharmaceutically acceptable salt thereof was present in tobacco-users in this study. If the doses used in the study were below the Eₘₐₓ for alpha 7 NNR activation, then this hypothetically greater brain concentration of (2S,3R)—N-(2-((3-pyridinyl)methyl)-1-azabicyclo[2.2.2]oct-3-yl)benzofuran-2-carboxamide or a pharmaceutically acceptable salt thereof in tobacco-users could result in a greater amount of receptor activation. Third, subjects in this study were asked to take (2S,3R)—N-(2-((3-pyridinyl)methyl)-1-azabicyclo[2.2.2]oct-3-yl)benzofuran-2-carboxamide pharmaceutically acceptable salt in the morning, at least 90 minutes before first tobacco intake (if smokers). Hence (2S,3R)—N-(2-((3-pyridinyl)methyl)-1-azabicyclo[2.2.2]oct-3-yl)benzofuran-2-carboxamide pharmaceutically acceptable salt may have acted upon sensitized alpha7 NNRs due to a night-time deprivation of nicotinic stimulation. Whether any of these or other factors underlie the greater effect of (2S,3R)—N-(2-((3-pyridinyl)methyl)-1-azabicyclo[2.2.2]oct-3-yl)benzofuran-2-carboxamide or a pharmaceutically acceptable salt thereof in tobacco users in this study will require further preclinical and clinical research. If, however, there is confirmation that (2S,3R)—N-(2-((3-pyridinyl)methyl)-1-azabicyclo[2.2.2]oct-3-yl)benzofuran-2-carboxamide or a pharmaceutically acceptable salt thereof or other alpha7 NNR agonists work primarily in tobacco-using patients with schizophrenia, this should not deter development of these compounds since the majority of patients with schizophrenia are smokers (Hughes et al., 1986; Goff et al., 1992; de Leon et al., 1995; Diwan et al., 1998; O'Carroll, 2000). One aspect of the invention includes a combination of (2S,3R)—N-(2-((3-pyridinyl)methyl)-1-azabicyclo[2.2.2]oct-3-yl)benzofuran-2-carboxamide or a pharmaceutically acceptable salt thereof.

There are a number of caveats that should be considered in the interpretation of this study. First, the numbers of tobacco users in the study was relatively small (46% of the enrolled population), and so the finding needs to be replicated in larger studies. Second, this study was conducted predominantly in India (approximately two-thirds of enrolled subjects), and therefore these findings need to be replicated in larger studies in other regions to demonstrate translation into other cultures and other ways of medical practice.

Nevertheless these findings are encouraging in supporting the role of alpha7 NNR agonists like (2S,3R)—N-(2-((3-pyridinyl)methyl)-1-azabicyclo[2.2.2]oct-3-yl)benzofuran-2-carboxamide or a pharmaceutically acceptable salt thereof to treat the residual phase of schizophrenia. The effect of a reliable treatment for these symptoms could be enormous for patients with schizophrenia, for the families and caregivers who help them, and for the society and economy that could benefit from these patient's return to more productive lives.

### Efficacy Results

Efficacy results are presented in Tables 4-8.

TABLE 4
Efficacy Results: Groton Maze Learning (GML)

| | | | | GML (Total Error) LOG | | |
|---|---|---|---|---|---|---|
| | | | | Mean Difference ± | | |
| | | | | SE between | One-sided 90% | |
| All Patients | | | | TC-5619 and | Confidence Interval | |
| | | Adjusted | Standard Error of | Placebo | for | One-sided |
| Visit | Treatment | Mean | Adjusted Mean | (Placebo-TC5619) | Mean Difference | P-Value |
| Week 1 | Placebo | −0.02 | 0.01 | | | |
| | TC-5619 | −0.01 | 0.01 | | | |
| | | | | −0.01 ± 0.02 | (−0.03, ∞) | 0.6709 |
| Week 4 | Placebo | 0.02 | 0.02 | | | |
| | TC-5619 | −0.03 | 0.02 | | | |
| | | | | 0.05 ± 0.02 | (0.02, ∞) | 0.0180 |
| Week 8 | Placebo | −0.02 | 0.02 | | | |
| | TC-5619 | −0.05 | 0.02 | | | |
| | | | | 0.02 ± 0.02 | (−0.00, ∞) | 0.1308 |
| Week 12 | Placebo | −0.02 | 0.02 | | | |
| | TC-5619 | −0.06 | 0.02 | | | |
| | | | | 0.04 ± 0.02 | (0.01, ∞) | 0.0405 |

Primary endpoint is statistically significant based on Hochberg adjustment for 3 comparisons (one-sided p < .1)

TABLE 5
Efficacy Results - SANS Total Score

| | | | | SANS TOTAL SCORE | | |
|---|---|---|---|---|---|---|
| | | | | Mean Difference ± | | |
| | | | | SE between | One-sided 90% | |
| All Patients | | | | TC-5619 and | Confidence Interval | |
| | | Adjusted | Standard Error of | Placebo | for | One-sided |
| Visit | Treatment | Mean | Adjusted Mean | (Placebo-TC5619) | Mean Difference | P-Value |
| Week 1 | Placebo | −0.92 | 0.93 | | | |
| | TC-5619 | −1.20 | 0.93 | | | |
| | | | | 0.28 ± 0.82 | (−0.77, ∞) | 0.3647 |
| Week 4 | Placebo | −2.74 | 1.11 | | | |
| | TC-5619 | −3.52 | 1.11 | | | |
| | | | | 0.78 ± 1.18 | (−0.74, ∞) | 0.2547 |
| Week 8 | Placebo | −5.55 | 1.25 | | | |
| | TC-5619 | −7.25 | 1.25 | | | |
| | | | | 1.71 ± 1.44 | (−0.14, ∞) | 0.1184 |
| Week 12 | Placebo | −6.23 | 1.39 | | | |
| | TC-5619 | −9.93 | 1.40 | | | |
| | | | | 3.70 ± 1.68 | (1.53, ∞) | 0.0147 |

TABLE 6
Efficacy Results - CGI Global Change

| | | | | CGI Global Change | | |
|---|---|---|---|---|---|---|
| | | | | Mean Difference ± | | |
| | | | | SE between | One-sided 90% | |
| All Patients | | | | TC-5619 and | Confidence Interval | |
| | | Adjusted | Standard Error of | Placebo | for | One-sided |
| Visit | Treatment | Mean | Adjusted Mean | (Placebo-TC5619) | Mean Difference | P-Value |
| Week 1 | Placebo | 3.80 | 0.15 | | | |
| | TC-5619 | 3.77 | 0.15 | | | |
| | | | | 0.03 ± 0.07 | (−0.06, ∞) | 0.3434 |
| Week 4 | Placebo | 3.64 | 0.15 | | | |
| | TC-5619 | 3.49 | 0.16 | | | |
| | | | | 0.15 ± 0.09 | (0.04, ∞) | 0.0485 |
| Week 8 | Placebo | 3.30 | 0.16 | | | |
| | TC-5619 | 3.22 | 0.16 | | | |
| | | | | 0.08 ± 0.12 | (−0.07, ∞) | 0.2533 |
| Week 12 | Placebo | 3.18 | 0.17 | | | |
| | TC-5619 | 3.03 | 0.17 | | | |
| | | | | 0.15 ± 0.14 | (−0.03, ∞) | 0.1502 |

TABLE 7
SGI - Cognition by Patient

| | | | | SGI-COGNITION TOTAL BY PATIENT | | |
|---|---|---|---|---|---|---|
| | | | | Mean Difference ± | | |
| | | | | SE between | One-sided 90% | |
| All Patients | | | | TC-5619 and | Confidence Interval | |
| | | Adjusted | Standard Error of | Placebo | for | One-sided |
| Visit | Treatment | Mean | Adjusted Mean | (Placebo-TC5619) | Mean Difference | P-Value |
| Week 1 | Placebo | 11.37 | 0.19 | | | |
| | TC-5619 | 11.18 | 0.19 | | | |
| | | | | 0.19 ± 0.20 | (−0.07, ∞) | 0.1757 |
| Week 4 | Placebo | 10.65 | 0.24 | | | |
| | TC-5619 | 10.60 | 0.24 | | | |
| | | | | 0.05 ± 0.28 | (−0.31, ∞) | 0.4282 |
| Week 8 | Placebo | 9.80 | 0.28 | | | |
| | TC-5619 | 9.79 | 0.29 | | | |
| | | | | 0.01 ± 0.36 | (−0.45, ∞) | 0.4914 |
| Week 12 | Placebo | 9.61 | 0.30 | | | |
| | TC-5619 | 8.95 | 0.31 | | | |
| | | | | 0.66 ± 0.39 | (−0.16, ∞) | 0.0455 |

SGI-Cog. Composed of 3 subscales (memory, attention and speed of thinking). Effect is driven by memory (p = 0.55) and attention (p = .021).

TABLE 8
CGI - SEVERITY

| | | | | CGI-SEVERITY | | |
|---|---|---|---|---|---|---|
| | | | | Mean Difference ± | | |
| | | | | SE between | One-sided 90% | |
| All Patients | | | | TC-5619 and | Confidence Interval | |
| | | Adjusted | Standard Error of | Placebo | for | One-sided |
| Visit | Treatment | Mean | Adjusted Mean | (Placebo-TC5619) | Mean Difference | P-Value |
| Week 1 | Placebo | −0.05 | 0.03 | | | |
| | TC-5619 | −0.03 | 0.03 | | | |
| | | | | −0.02 ± 0.03 | (−0.07, ∞) | 0.7588 |
| Week 4 | Placebo | −0.09 | 0.05 | | | |
| | TC-5619 | −0.06 | 0.05 | | | |
| | | | | −0.02 ± 0.06 | (−0.10, ∞) | 0.6737 |
| Week 8 | Placebo | −0.24 | 0.05 | | | |
| | TC-5619 | −0.23 | 0.05 | | | |
| | | | | −0.01 ± 0.06 | (−0.09, ∞) | 0.5440 |
| Week 12 | Placebo | −0.29 | 0.07 | | | |
| | TC-5619 | −0.31 | 0.07 | | | |
| | | | | 0.01 ± 0.09 | (−0.11, ∞) | 0.4495 |

SGI-Cog. Composed of 3 subscales (memory, attention and speed of thinking). Effect is driven by memory (p = .055) and attention (p = .021).

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A method for the treatment of cognitive dysfunction in schizophrenia comprising administering (2S,3R)—N-(2-((3-pyridinyl)methyl)-1-azabicyclo[2.2.2]oct-3-yl)benzofuran-2-carboxamide or a pharmaceutically acceptable salt thereof to a patient in need thereof.

2. (canceled)

3. (canceled)

4. The method of claim 1, wherein the treatment provides an improvement in executive function.

5. The method of claim 1, wherein the treatment provides an improvement in memory.

6. The method of claim 1, wherein the treatment provides an improvement in attention.

7. A method for the treatment of negative symptoms of schizophrenia comprising administering (2S,3R)—N-(2-((3-pyridinyl)methyl)-1-azabicyclo[2.2.2]oct-3-yl)benzofuran-2-carboxamide or a pharmaceutically acceptable salt thereof to a patient in need thereof.

8. (canceled)

9. (canceled)

10. A method for the treatment of residual phase schizophrenia comprising administering (2S,3R)—N-(2-((3-pyridinyl)methyl)-1-azabicyclo[2.2.2]oct-3-yl)benzofuran-2-carboxamide or a pharmaceutically acceptable salt thereof to a patient in need thereof.

11. (canceled)

12. (canceled)

13. A pharmaceutical composition for the treatment of one or more of cognitive dysfunction in schizophrenia, negative symptoms of schizophrenia, or residual phase schizophrenia comprising (2S,3R)—N-(2-((3-pyridinyl)methyl)-1-azabicyclo[2.2.2]oct-3-yl)benzofuran-2-carboxamide or a pharmaceutically acceptable salt thereof and one or more pharmaceutically acceptable excipients.

14. The pharmaceutical composition of claim 13, wherein the composition further comprises one or more antipsychotic medications, anti-depressants, or mood stabilizers.

15. The pharmaceutical composition of claim 14, wherein the one or more antipsychotic medication is one or more of: Stelazine (Trifluoperazine), Flupenthixol (Fluanxol), Loxapine (Loxapac, Loxitane), Perphenazine (Etrafon, Trilafon), Chlorpromazine (Thorazine), Haldol (Haloperidol), Prolixin (Fluphenazine Decanoate, Modecate, Permitil), Aripiprazole (Abilify), Clozaril (clozapine), Geodon (ziprasidone), Risperdal (resperidone), Seroquel (Quetiapine), Zyprexa (olanzapine), or an agonist of the nicotinic α7 receptor.
